(19) United States
(12) Patent Application Publication
(10) Pub. No.: US 2025/0185965 A1
(43) Pub. Date: Jun. 12, 2025

(54) DUAL-WALL HEAT SHRINK FORMING MOLD AND NEURODIAGNOSTIC NEEDLE ELECTRODE PAIR HUB FORMED THEREBY

(71) Applicant: ROCWorks, LLC, Prosperity, SC (US)

(72) Inventor: Brett Lane Netherton, Prosperity, SC (US)

(73) Assignee: ROCWorks, LLC, Prosperity, SC (US)

(21) Appl. No.: 18/973,737

(22) Filed: Dec. 9, 2024

Related U.S. Application Data
(60) Provisional application No. 63/607,185, filed on Dec. 7, 2023.

(51) Int. Cl. A61B 5/262

(52) U.S. Cl. CPC A61B 5/262; A61B 2562/125

(57) ABSTRACT

A neurodiagnostic needle electrode pair assembly is provided that includes a pair of needle electrodes and a recovered dual-wall heat shrink tube. The needle electrodes are connected to a pair of leadwires by respective electrical connections between proximal ends of the needle electrodes and distal ends of the leadwires. The recovered dual-wall heat shrink tube covers the proximal ends of the needle electrodes, the respective electrical connections and the distal ends of the leadwires. The recovered dual-wall heat shrink tube includes an inner lining of adhesive that secures the needle electrodes in a fixed, spaced apart relationship. A mold for a neurodiagnostic needle electrode pair assembly and a method of manufacturing a neurodiagnostic needle electrode pair assembly are also provided.

### CROSS-REFERENCE TO RELATED APPLICATION(S)

The present application claims priority to U.S. Provisional Patent Application No. 63/607,185, entitled: Dual-Wall Heat Shrink Forming Mold and Neurodiagnostic Needle Electrode Pair Hub Formed Thereby, filed on Dec. 7, 2023, the content of which is hereby incorporated by reference in its entirety.

### TECHNOLOGICAL FIELD

The present disclosure relates generally to the process of forming adhesive-lined thermoplastic shrinkable tubing (dual-wall heat shrink) to specific configurations and shapes during the thermal recovery process. It may pertain to the formed hub of a neurodiagnostic needle electrode pair and manufacture of the same.

### BACKGROUND

Dual-wall heat shrink may form with inconsistencies in outer shape and therefore inconsistency of adhesive lining filling during the manufacturing process. Manufacturing techniques that create consistency in outer shape of the recovered dual-wall heat shrink and consistency in filling adhesive lining voids in the assembly are desirable.

An example of desirable outcome is creating a neurodiagnostic needle electrode pair with a formed hub which may include using dual-wall heat shrink as an exoskeleton over two extrusions in abutment. dual-wall heat shrink with adhesive as the inner lining is desirable as the exoskeleton of the formed hub for increased strength and fluid resistance.

Another example of desirable outcome is creating a neurodiagnostic needle electrode pair with a formed hub which may include using dual-wall heat shrink as an exoskeleton and the adhesive of the dual-wall heat shrink as electrical insulation and structural fill around the needle/electrical securement/leadwire subassembly of the neurodiagnostic needle electrode pair.

It may be beneficial for the needle electrode pair used to perform the physiologic recording to be ergonomic to the clinician placing the needle electrodes. The handhold grip portion of any clinically used needle electrode may typically be referred to as a “hub.” Other names may include but are not limited to a “grip,” “hold,” or other such similar names. For paired needle electrodes that may require a specific inter-needle distance, the hub may facilitate consistency in the needle spacing as well as provide the clinician with a comfortable hub. Also, because the electrical recordings (or likewise stimulation) require electrical exposure only at the needle tip in contact with the patient tissue, the hub of the needle electrode pair may constitute a barrier against fluids that could compromise the electrical integrity of the needle electrode pair by an electrical short circuit inside of the hub.

The techniques used to manufacture paired needle electrodes have included forming the needle electrode pair via injection molding a rigid plastic hub to cover the electrical connections. The electrical connections that are covered by the plastic hub typically include either a solder joint or mechanical crimp to electrically connect the needle with the electrode leadwire. The electrode leadwire then electrically connects the electrode patient attachment with the neurodiagnostic electrical instrumentation equipment. The process of injection molding is well established as an effective way of producing such medical devices. It is also a relatively expensive medical device production technique. Alternative manufacturing techniques that are more cost effective are desired.

### BRIEF SUMMARY

In example applications described herein, a method for forming dual-wall heat shrink into desired shapes and configurations during the thermal recovery process is disclosed. Examples of this may include using a dual-wall heat shrink forming mold to form various dual-wall heat shrink hubs of neurodiagnostic needle electrode pairs. The present disclosure includes, without limitation, the following example implementations.

Some example implementations provide a neurodiagnostic needle electrode pair assembly comprising: a pair of needle electrodes connected to a pair of leadwires by respective electrical connections between proximal ends of the needle electrodes and distal ends of the leadwires; and a recovered dual-wall heat shrink tube covering the proximal ends of the needle electrodes, the respective electrical connections and the distal ends of the leadwires, the recovered dual-wall heat shrink tube including an inner lining of adhesive that secures the needle electrodes in a fixed, spaced apart relationship.

Some example implementations provide a mold for a neurodiagnostic needle electrode pair assembly that includes a pair of needle electrodes connected to a pair of leadwires by respective electrical connections between proximal ends of the needle electrodes and distal ends of the leadwires, the mold comprising: a mold base including a base molding cavity defined to receive a first portion of a dual-wall heat shrink tube covering the proximal ends of the needle electrodes, the respective electrical connections and the distal ends of the leadwires; and a mold top configured to mate with the mold base, the mold top including a top molding cavity defined to receive a second portion of the dual-wall heat shrink when the mold top mold is mated with the mold base, wherein when the mold top and the mold base are mated during a thermal recovery process of the dual-wall heat shrink tube in which the adhesive is in a molten or semi-molten state, the mold top and the mold base apply a force that creates pressure on the adhesive, and the pressure forces the adhesive to flow outward and creates outward hydraulic pressure on the dual-wall heat shrink tube to form in an outward direction against inside surfaces of the top molding cavity and the base molding cavity.

Some example implementations provide a method of manufacturing a neurodiagnostic needle electrode pair assembly that includes a pair of needle electrodes connected to a pair of leadwires by respective electrical connections between proximal ends of the needle electrodes and distal ends of the leadwires, the method comprising: receiving a first portion of a dual-wall heat shrink tube in a base molding cavity of a mold base, the dual-wall heat shrink tube covering the proximal ends of the needle electrodes, the respective electrical connections and the distal ends of the leadwires; and mating a mold top with the mold base during a thermal recovery process of the dual-wall heat shrink tube in which the adhesive is in a molten or semi-molten state, the mold top including a top molding cavity defined to receive a second portion of the dual-wall heat shrink when the mold top is mated with the mold base, wherein when the mold top and the mold base are mated, the mold top and the mold base apply a force that creates pressure on the adhesive, and the pressure forces the adhesive to flow outward and creates outward hydraulic pressure on the dual-wall heat shrink tube to form in an outward direction against inside surfaces of the top molding cavity and the base molding cavity.

These and other features, aspects, and advantages of the present disclosure will be apparent from a reading of the following detailed description together with the accompanying figures, which are briefly described below. The present disclosure includes any combination of two, three, four or more features or elements set forth in this disclosure, regardless of whether such features or elements are expressly combined or otherwise recited in a specific example implementation described herein. The present disclosure is intended to be read holistically such that any separable features or elements of the disclosure, in any of its aspects and example implementations, should be viewed as combinable unless the context of the disclosure clearly dictates otherwise.

It will therefore be appreciated that this Brief Summary is provided merely for purposes of summarizing some example implementations so as to provide a basic understanding of some aspects of the disclosure. Accordingly, it will be appreciated that the above described example implementations are merely examples and should not be construed to narrow the scope or spirit of the disclosure in any way. Other example implementations, aspects and advantages will become apparent from the following detailed description taken in conjunction with the accompanying figures which illustrate, by way of example, the principles of some described example implementations.

### BRIEF DESCRIPTION OF THE FIGURE(S)

Having thus described example implementations of the disclosure in general terms, reference will now be made to the accompanying figures, which are not necessarily drawn to scale, and wherein:

FIGS. 1A and 1B illustrate a neurodiagnostic needle electrode pair with an injection molded hub and two neurodiagnostic needle electrode pairs with formed dual-wall heat shrink hubs, according to example implementations;

FIGS. 2A and 2B illustrate a neurodiagnostic needle electrode pair subassembly without and with a segment of unrecovered dual-wall heat shrink, according to some example implementations;

FIGS. 3A and 3B illustrate a formed dual-wall heat shrink hub with consistent outer shape formed over a neurodiagnostic needle electrode pair subassembly, according to some example implementations;

FIG. 4 illustrates mold base, hinge pin, needle plate, molding plate, and leadwire plate, assembled to form a dual-wall heat shrink forming mold, according to example implementations;

FIGS. 5A, 5B, 5C, 5D, 5E, 5F, 5G, 5H, 5I, 5J, 5K, 5L, 5M, 5N, 50, 5P, 5Q, and 5R illustrate a dual-wall heat shrink forming mold in use to form a recovered dual-wall heat shrink hub, according to example implementations;

FIG. 6 illustrates a cross-sectional view of a neurodiagnostic needle electrode pair subassembly with recovered dual-wall heat shrink exoskeleton formed using a dual-wall heat shrink forming mold, according to example implementations;

FIG. 7 illustrates a dual-wall heat shrink hub formed using a dual-wall heat shrink forming mold, according to example implementations;

FIGS. 8A and 8B illustrate a dual-wall heat shrink forming mold, comprised of a base holding fixture, a needle cap, a leadwire cap, a mold top, and a mold base, according to example implementations;

FIGS. 9A, 9B, 9C and 9D illustrate multiple views of the base holding fixture, according to example implementations;

FIG. 10 illustrates the needle cap, according to example implementations;

FIG. 11 illustrates the leadwire cap, according to example implementations;

FIGS. 12A and 12B illustrate multiple views of the mold top and mold base, according to example implementations;

FIGS. 13A, 13B, 13C, 13D, 13E, 13F, 13G, 13H, 13I, 13J, 13K, 13L, 13M, 13N, 130, 13P, 13Q, 13R, 13S, 13T, 13U and 13V illustrate a dual-wall heat shrink forming mold in use to form a recovered dual-wall heat shrink hub, according to example implementations;

FIG. 14 illustrates a dual-wall heat shrink hub formed using a dual-wall heat shrink forming mold, according to example implementations;

FIGS. 15A and 15B illustrate a dual-wall heat shrink forming mold, comprised of a base holding fixture, a needle cap, a leadwire cap, a mold top which includes thermally conducting fins, a mold base which includes thermally conducting fins, and a hinge pin, according to example implementations;

FIGS. 15C, 15D, 15E, 15F, 15G, 15H and 15I illustrate a dual-wall heat shrink forming mold in use to form a recovered dual-wall heat shrink hub, according to example implementations;

FIG. 16 illustrates various molding cavities that may be created when mold tops and mold bases are in fully mated position, according to example implementations; and

FIGS. 17A, 17B, 17C, 17D and 17E illustrate example alternative modifications and other implementations of shapes and configurations of formed dual-wall heat shrink hubs, according to example implementations.

### DETAILED DESCRIPTION

Some implementations of the present disclosure will now be described more fully hereinafter with reference to the accompanying figures, in which some, but not all implementations of the disclosure are shown. Indeed, various implementations of the disclosure may be embodied in many different forms and should not be construed as limited to the implementations set forth herein; rather, these example implementations are provided so that this disclosure will be thorough and complete, and will fully convey the scope of the disclosure to those skilled in the art. Like reference numerals refer to like elements throughout.

Unless specified otherwise or clear from context, references to first, second or the like should not be construed to imply a particular order. A feature described as being above another feature (unless specified otherwise or clear from context) may instead be below, and vice versa; and similarly, features described as being to the left of another feature else may instead be to the right, and vice versa. Also, while reference may be made herein to quantitative measures, values, geometric relationships or the like, unless otherwise stated, any one or more if not all of these may be absolute or approximate to account for acceptable variations that may occur, such as those due to engineering tolerances or the like.

As used herein, unless specified otherwise or clear from context, the “or” of a set of operands is the “inclusive or” and thereby true if and only if one or more of the operands is true, as opposed to the “exclusive or” which is false when all of the operands are true. Thus, for example, “[A] or [B]” is true if [A] is true, or if [B] is true, or if both [A] and [B] are true. Further, the articles “a” and “an” mean “one or more,” unless specified otherwise or clear from context to be directed to a singular form. Furthermore, it should be understood that unless otherwise specified, the terms “data,” “content,” “digital content,” “information,” and similar terms may be at times used interchangeably.

Some example implementations are described herein of manufacturing techniques to form dual-wall heat shrink to specific configurations and shapes during the thermal recovery process. Further example implementations are described herein using a dual-wall heat shrink forming mold to produce the formed hub of a neurodiagnostic needle electrode pair that does not require the conventional injection molding process yet performs the required functions of the hub and is cost effective.

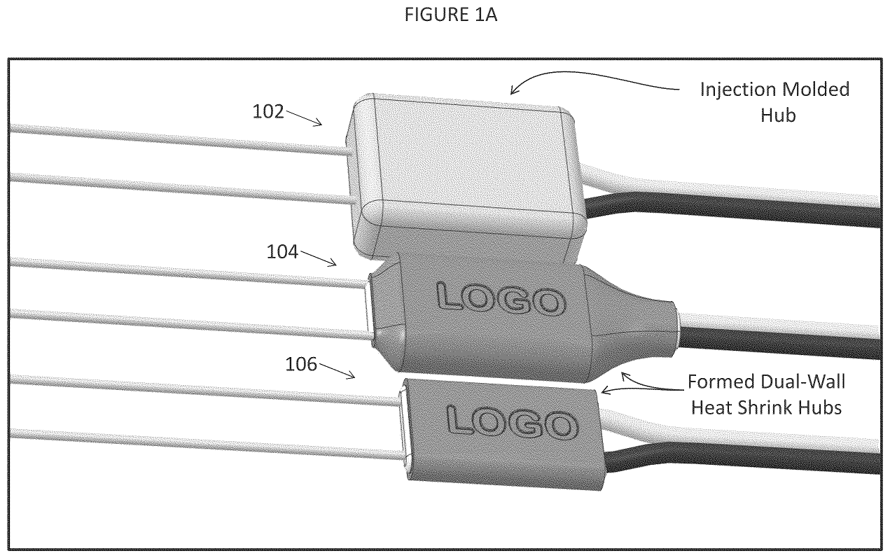

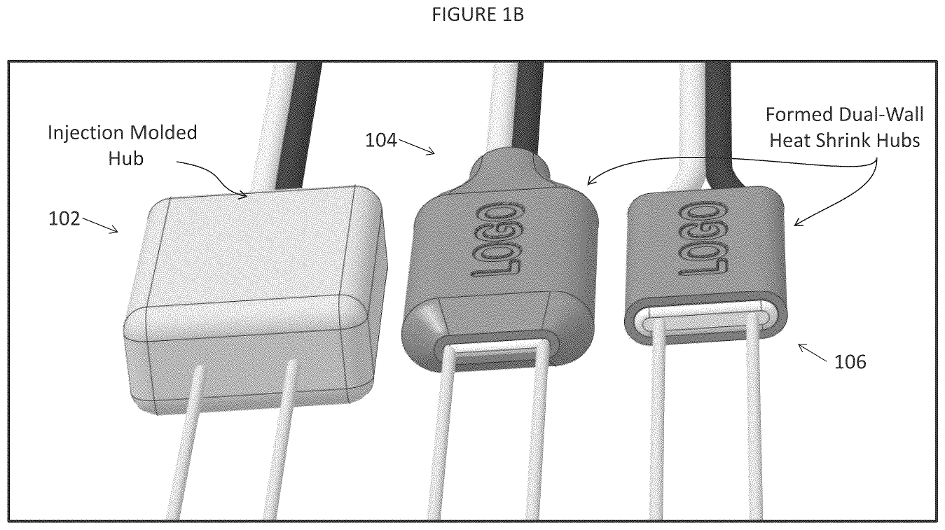

Some example implementations provide an alternative to more expensive injection molding processes that yields the desirable outcome of forming desired shapes and configurations of recovered dual-wall heat shrink as highlighted in FIGS. 1A and 1B, showing a neurodiagnostic needle electrode pair 102 with an injection molded hub and two neurodiagnostic needle electrode pairs 104, 106 with formed dual-wall heat shrink hubs. In some examples, a formed dual-wall heat shrink hub for a neurodiagnostic needle electrode pair is manufactured while maintaining the same salient structural characteristics/requirements including: a fluid resistant barrier covering the electrical connections between the needle and the leadwire; and needle separation and inter-needle rigidity. Because of their small wire diameter, the needle electrodes may be inherently flexible, however, at their base, they may be manufactured to be well-held in parallel position.

In some examples, dual-wall heat shrink may be preferred for purposes of forming the exoskeleton cover of the neurodiagnostic needle electrode pair due to the inner lining of adhesive such as adhesive glue that melts and flows near the temperature required to recover the dual-wall heat shrink outer layer. As the outer layer recovers, molten or semi-molten adhesive seeks to encapsulate the underlying structures, thereby enhancing resistance against fluid ingress and providing enhanced securement against and of the leadwire pair and other components of the subassembly. As described herein, reference to molten adhesive may indicate the adhesive in a molten or semi-molten state, unless otherwise indicated.

Some example implementations described herein may pertain to creating the formed hub of a neurodiagnostic needle electrode pair using dual-wall heat shrink as an exoskeleton, and manufacture of the same. dual-wall heat shrink with adhesive as the inner lining may be desirable for increased strength and fluid resistance. Some example implementations are described herein of manufacturing techniques to form dual-wall heat shrink to specific configurations and shapes during the thermal recovery process. Further example implementations are described herein using a dual-wall heat shrink forming mold to produce the formed hub of a neurodiagnostic needle electrode pair that does not require the conventional injection molding process yet performs the required functions of the hub and is cost effective. The example implementations provide an alternative to more expensive injection molding processes that yields a conventional hub.

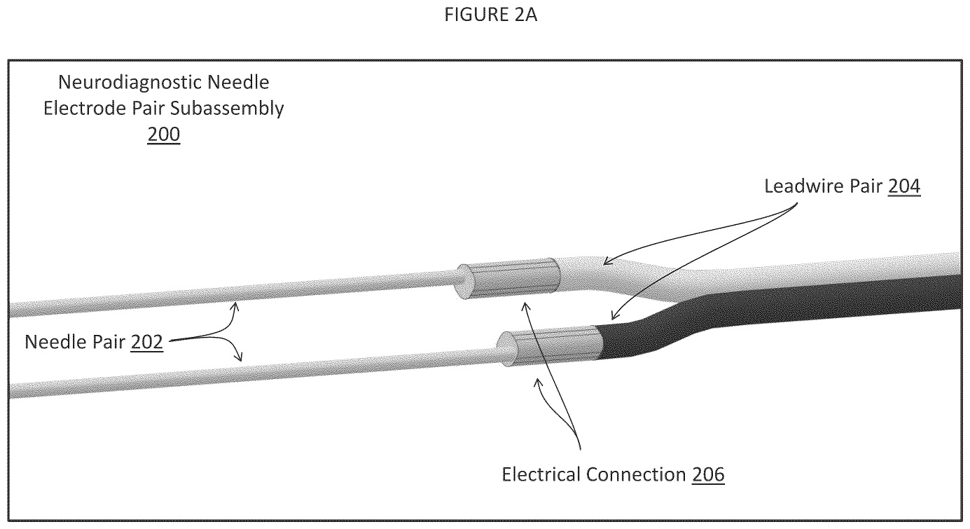

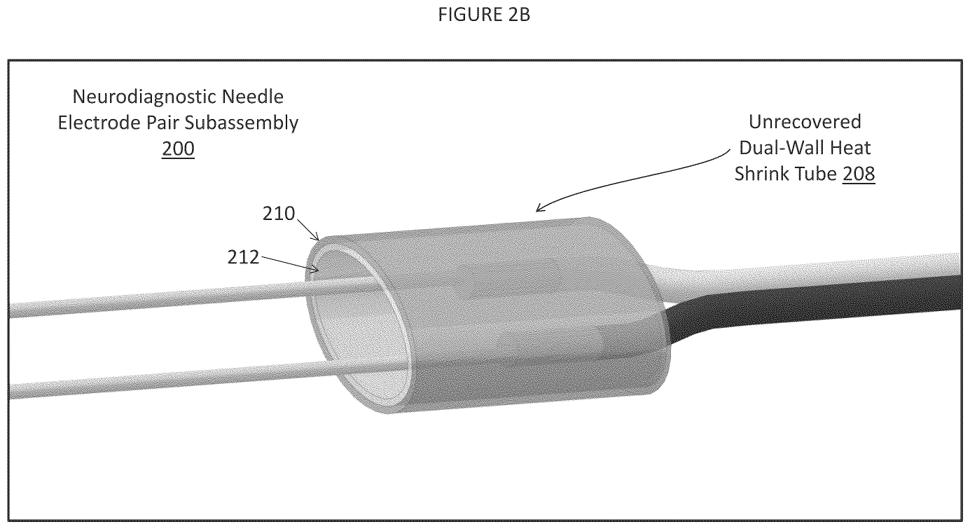

As shown in FIG. 2A, in some examples, a neurodiagnostic needle electrode pair subassembly 200 may include a needle electrode pair 202, a leadwire pair 204, and a securement such as electrical connections 206 performing electrical and mechanical connection of the needle to the leadwire. Example securement techniques could be mechanical crimp, solder joint, or others. FIG. 2B illustrates a segment of unrecovered dual-wall heat shrink (also referred to as a dual-wall heat shrink tube 208) in place over a neurodiagnostic needle electrode pair subassembly (shown in FIG. 2A). The dual-wall heat shrink tube includes an outer layer 210 and an inner lining of adhesive 212, such as adhesive glue.

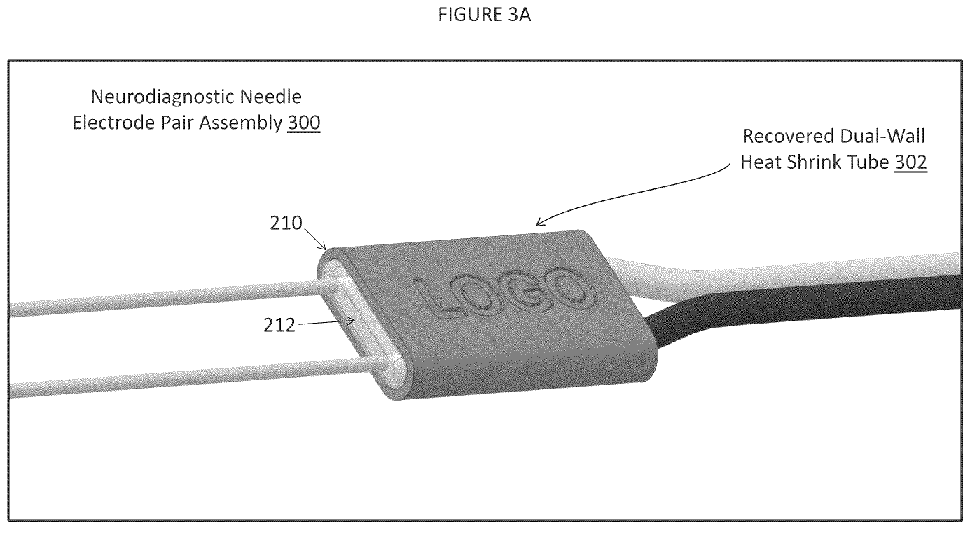

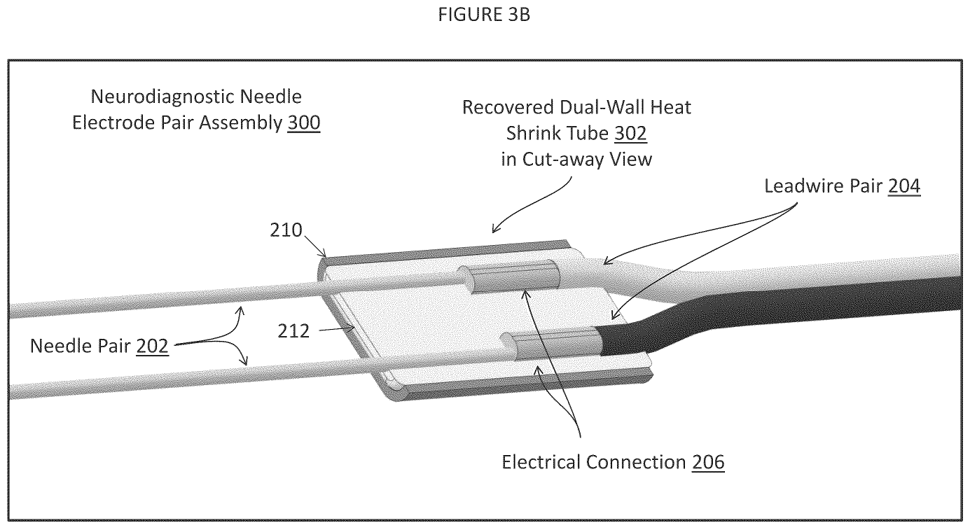

FIGS. 3A and 3B illustrate the neurodiagnostic needle electrode pair assembly 300 that includes a recovered dual-wall heat shrink tube 302 (also referred to as a formed dual-wall heat shrink hub, or more simply a formed hub) with consistent outer shape. The recovered dual-wall heat shrink tub is formed over a neurodiagnostic needle electrode pair subassembly 200 that includes a needle electrode pair 202, a leadwire pair 204, and electrical connections 206 of the needle to the leadwire (shown in FIG. 2A), according to some examples. FIG. 3B is a cross-sectional view of FIG. 3A which illustrates the adhesive of the dual-wall heat shrink encompassing the underlying structures of the neurodiagnostic needle electrode pair subassembly. The recovered dual-wall heat shrink tube 302 may in some examples have a non-circular cross section.

The outer layer 210 of the recovered dual-wall heat shrink tube 302 defines openings at opposing ends of the dual-wall heat shrink tube through which the needle electrodes 202 and the leadwires 204 extend. And in some examples, the adhesive 212 (adhesive glue) fills the openings around the needle electrodes and the leadwires.

As shown, then, the neurodiagnostic needle electrode pair assembly 300 includes a pair of needle electrodes 202 connected to a pair of leadwires 204 by respective electrical connections 206 between proximal ends of the needle electrodes and distal ends of the leadwires. The assembly also includes a recovered dual-wall heat shrink tube 302 covering the proximal ends of the needle electrodes, the respective electrical connections and the distal ends of the leadwires. The recovered dual-wall heat shrink tube includes an inner lining of adhesive 212 that secures the needle electrodes in a fixed, spaced apart relationship. And the inner lining of adhesive may also secure the respective electrical connections and the distal ends of the leadwires in a fixed, spaced apart relationship.

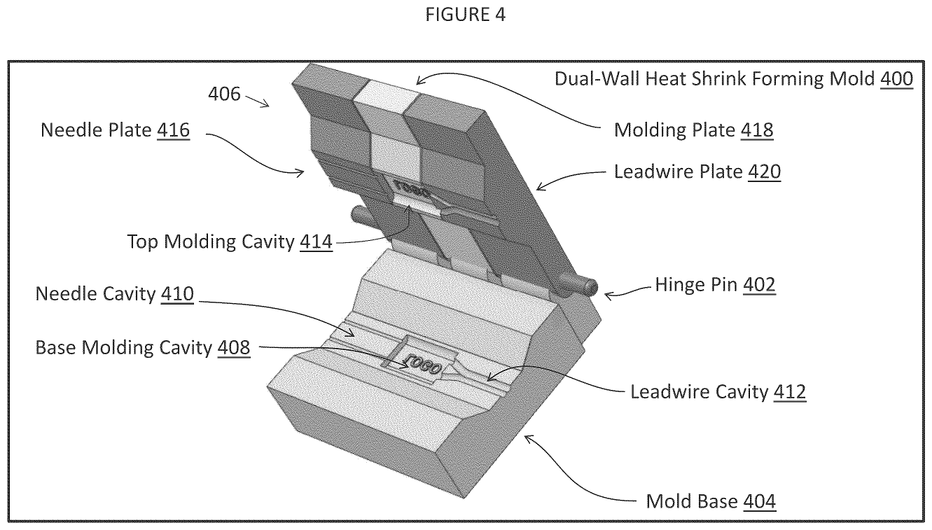

FIG. 4 illustrates an example implementation of a dual-wall heat shrink forming mold 400 suited for producing a formed dual-wall heat shrink hub on a neurodiagnostic needle electrode pair assembly 300. As described above, the neurodiagnostic needle electrode pair assembly may include a recovered dual-wall heat shrink tube 302 as an exoskeleton, and the adhesive 212 of the dual-wall heat shrink as electrical insulation and structural fill around the neurodiagnostic needle electrode pair subassembly 200. This example implementation of a dual-wall heat shrink forming mold includes a hinge pin 402 connecting a mold base 404 (also referred to as a forming mold base) with a mold top 406 (also referred to as forming mold top).

The mold base 404 includes a base molding cavity 408 defined to receive a first portion of a dual-wall heat shrink tube, and may also include needle cavities 410 defined to receive and hold the needle electrodes 202, and/or leadwire cavities 412 defined to receive and hold the leadwires 204. The mold top 406 may likewise include one or more cavities, including a top molding cavity 414. The base molding cavity and the top molding cavity may form a molding cavity when the mold base and the mold top are mated.

As also shown, the mold top 406 may include a needle plate 416 (also referred to as a needle hinge plate), a molding plate 418 (also referred to as a molding hinge plate), and/or a leadwire plate 420 (also referred to as a leadwire hinge plate). The needle plate may rotate down against the mold base 404 to tightly bind the needle electrodes 202 against movement in the needle cavity 410. The leadwire plate may rotate down against the mold base to tightly bind the leadwires 204 against movement in the leadwire cavity 412. The molding plate 418 may rotate down against the mold base to form the molding cavity.

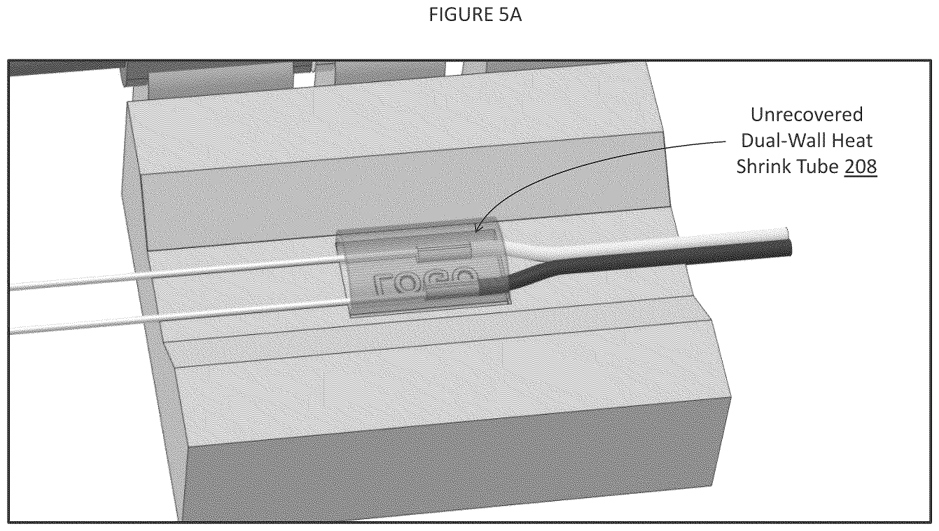

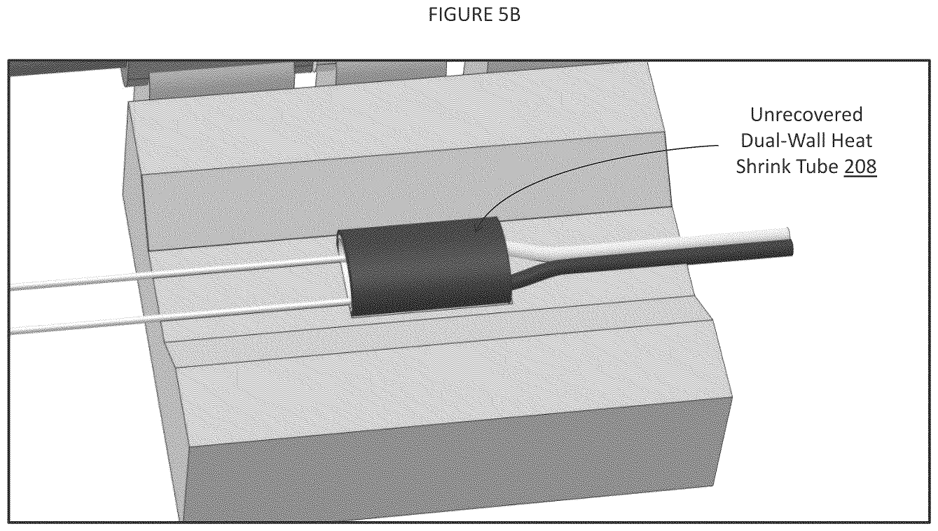

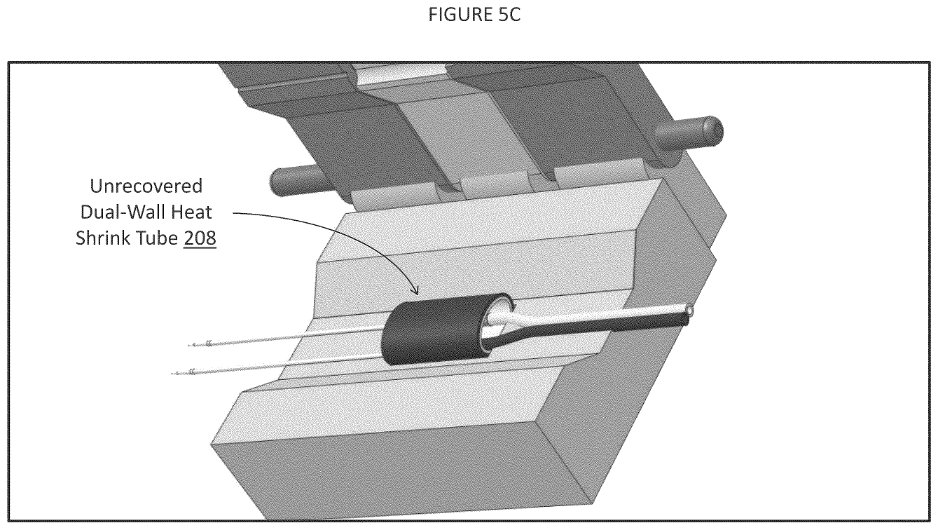

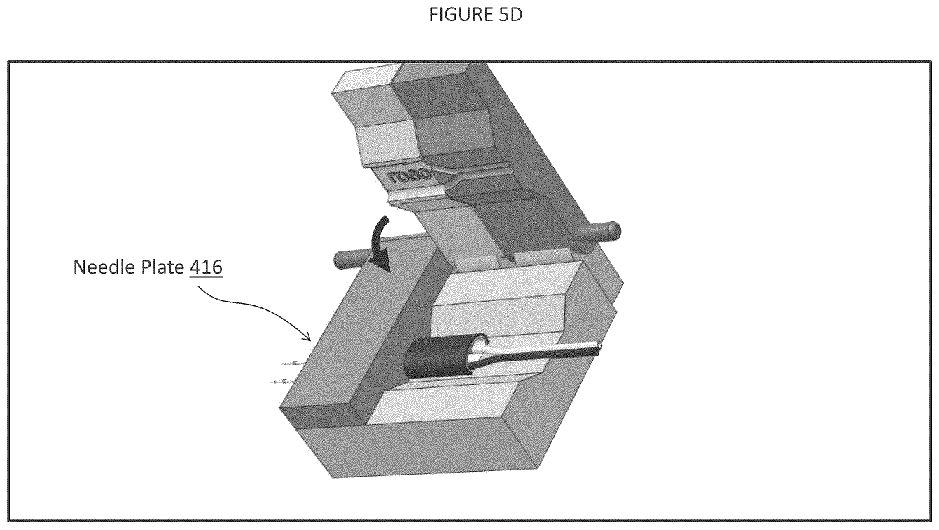

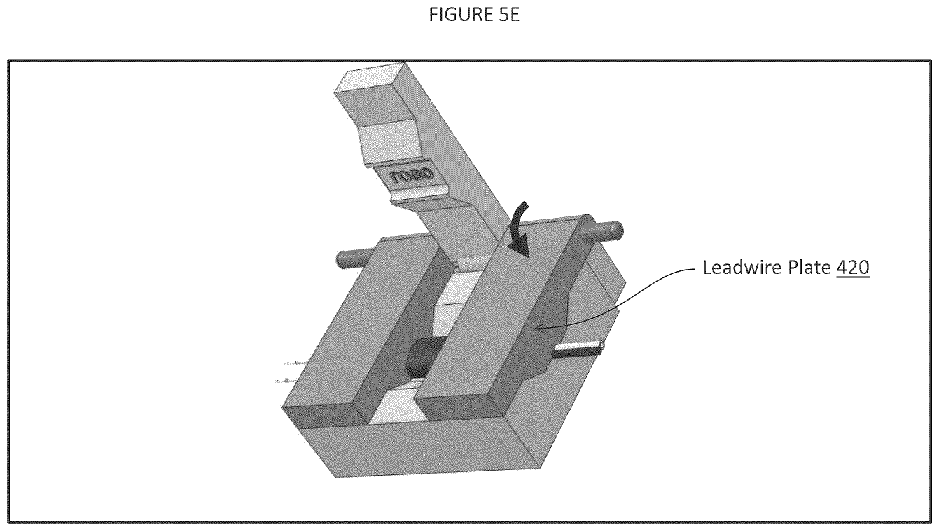

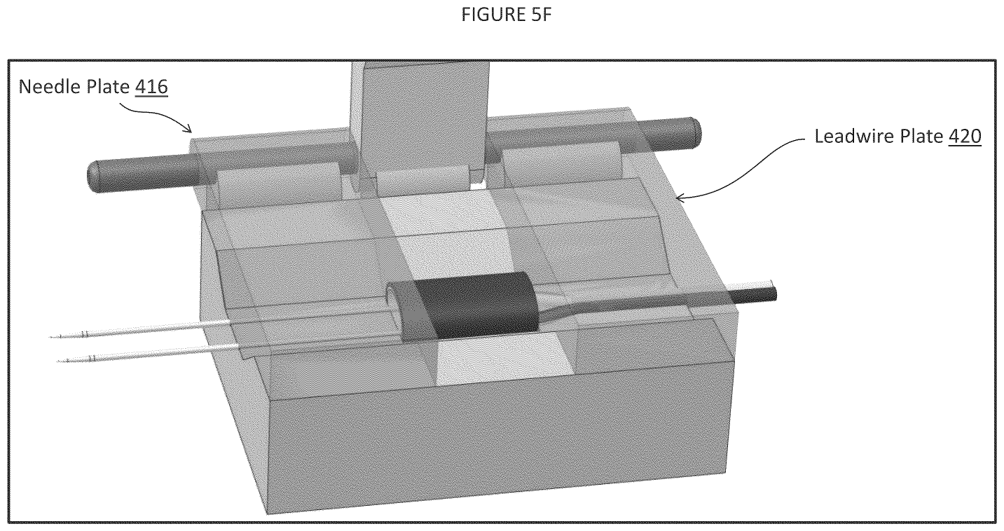

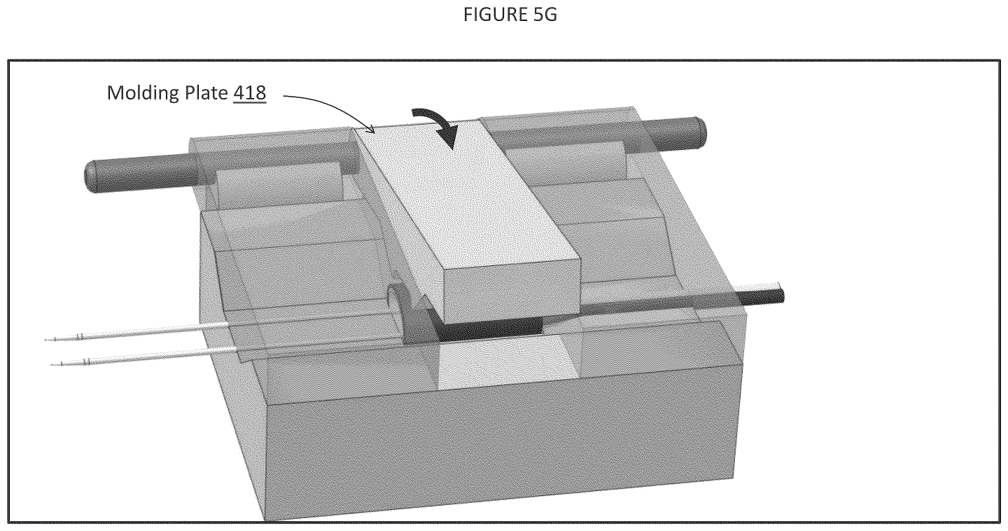

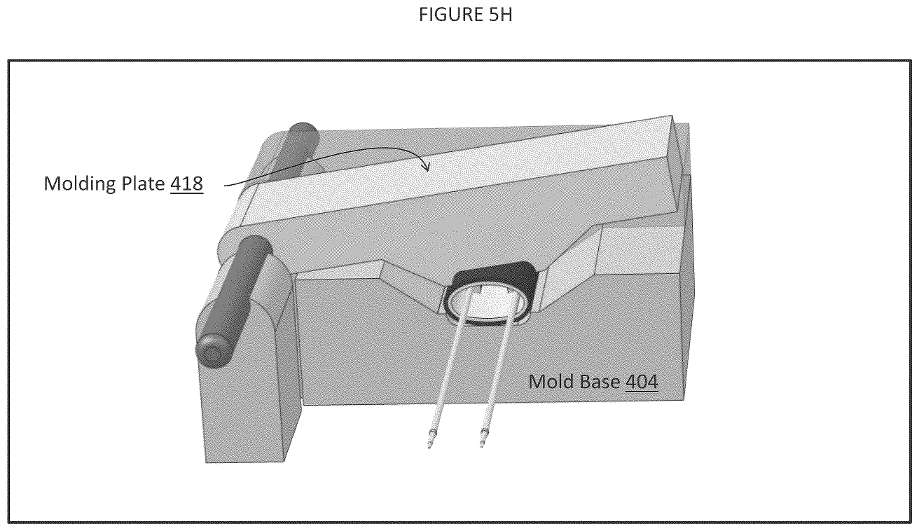

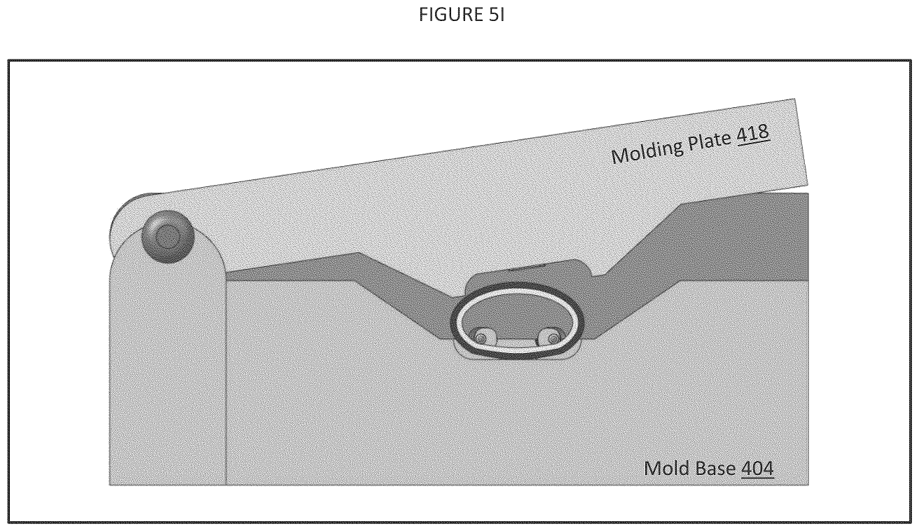

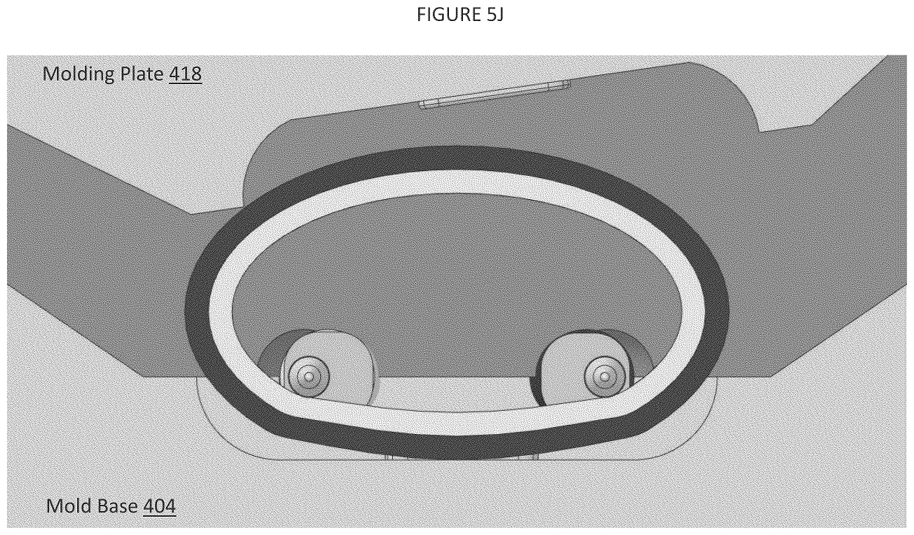

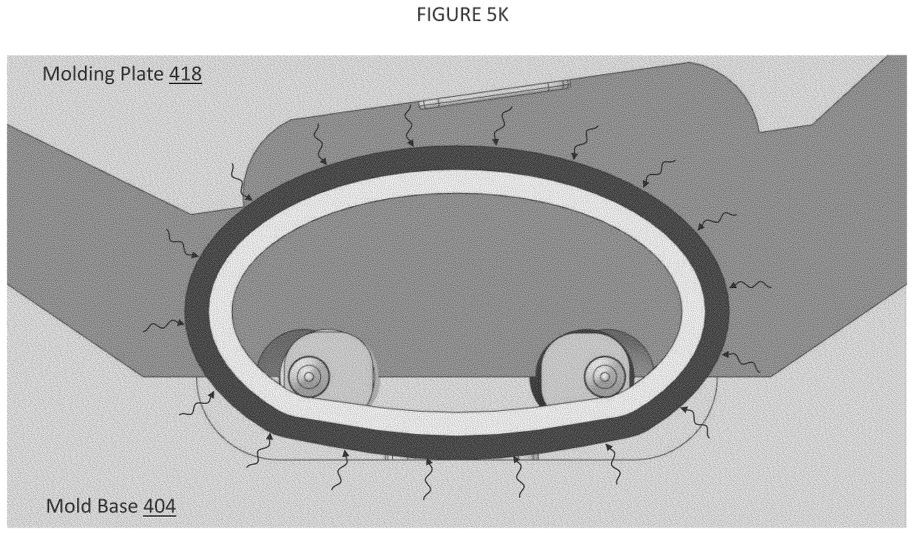

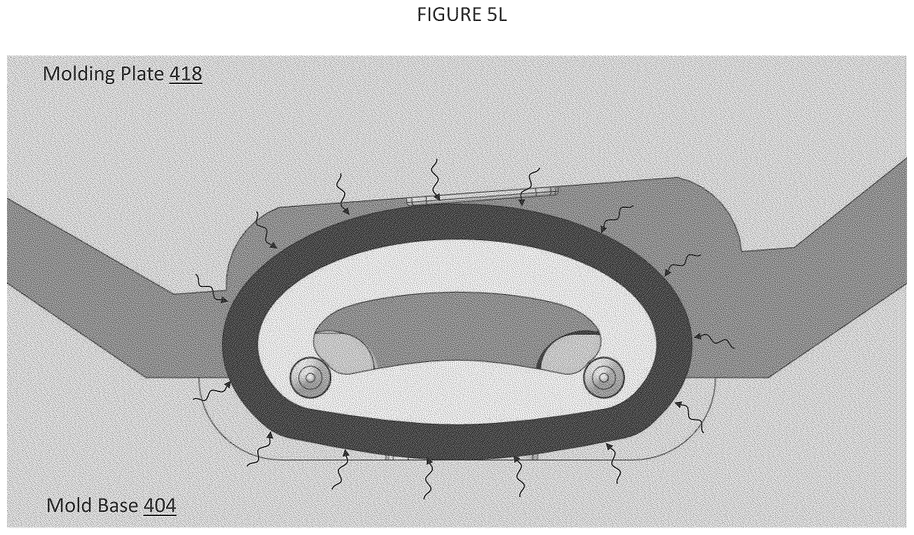

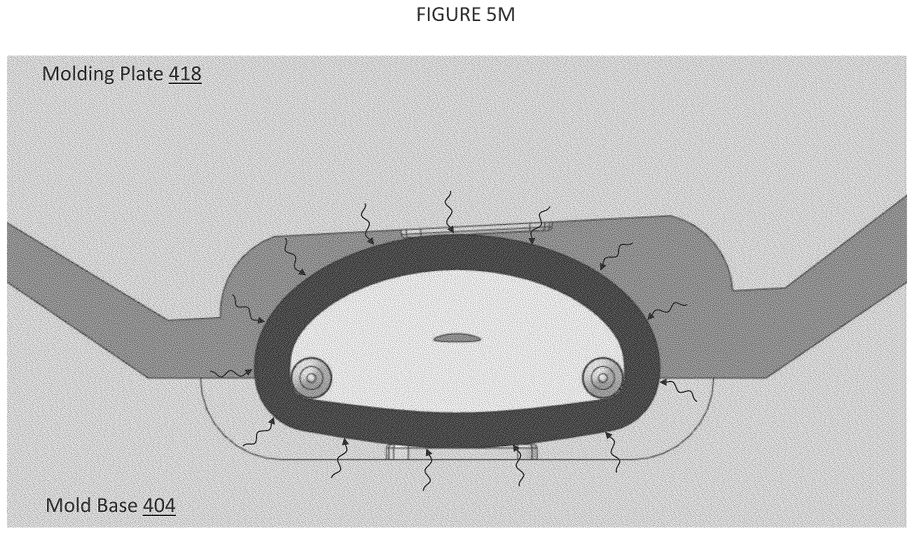

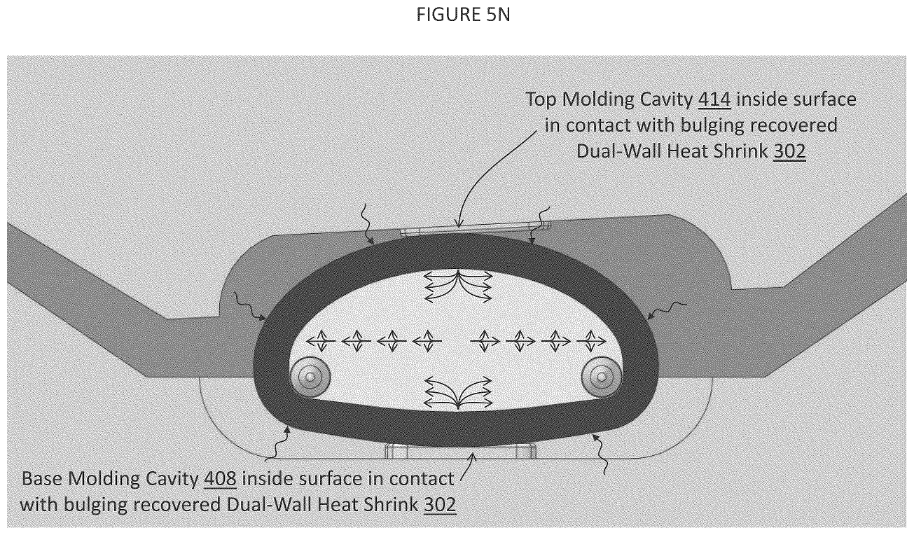

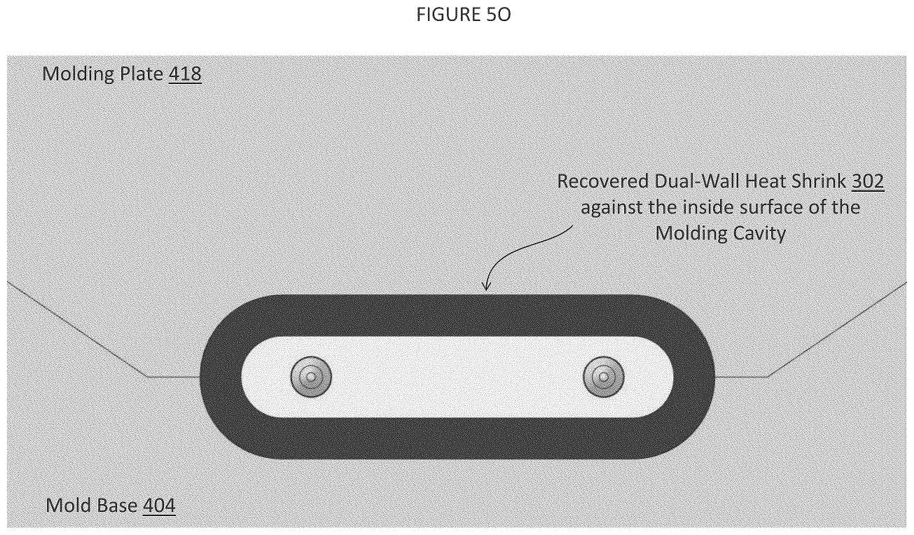

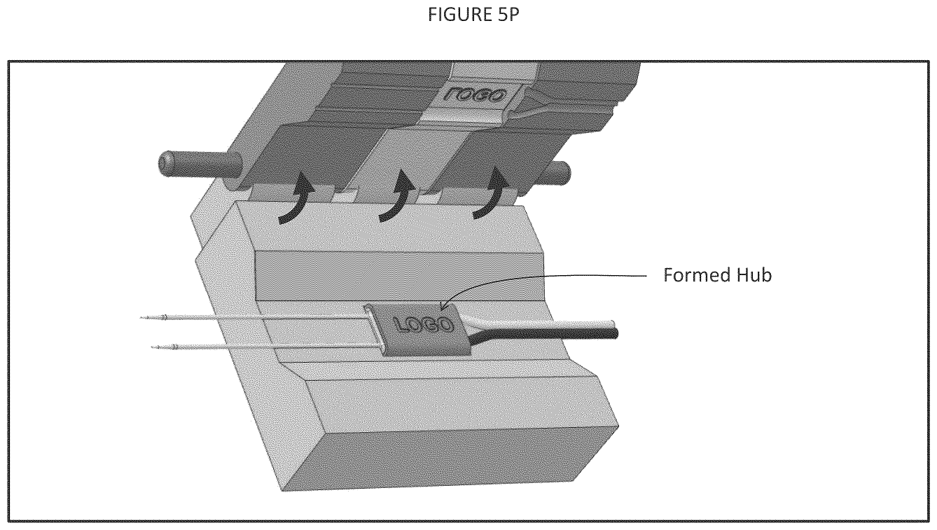

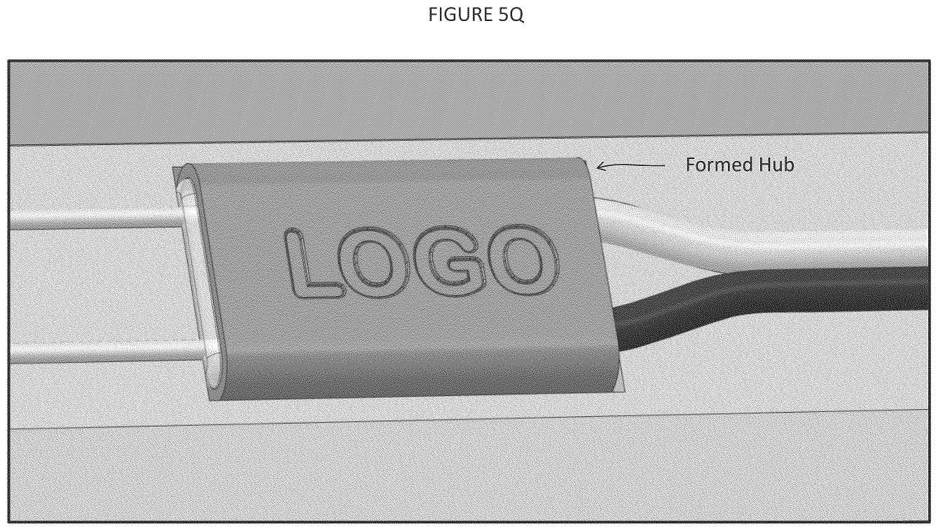

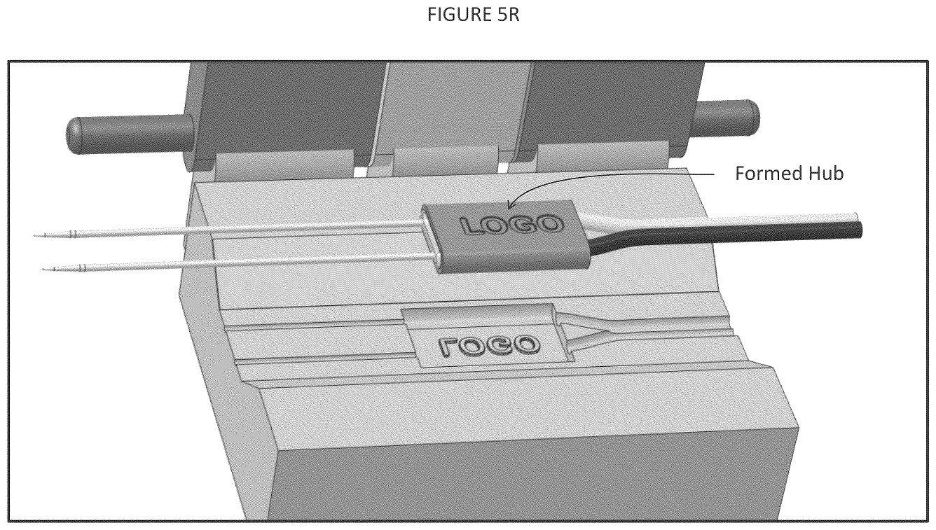

FIGS. 5A, 5B, 5C, 5D, 5E, 5F, 5G, 5H, 5I, 5J, 5K, 5L, 5M, 5N, 50, 5P, 5Q and 5R show steps of a method of manufacturing a neurodiagnostic needle electrode pair assembly 300 including a recovered dual-wall heat shrink tube 302 (formed dual-wall heat shrink hub) on a neurodiagnostic needle electrode pair subassembly 200, according to some example implementations. As shown, the method of some examples utilizes an implementation of the dual-wall heat shrink forming mold 400 as shown in FIGS. 4A and 4B.

FIGS. 5A, 5B and 5C illustrate a first step may include placing an unrecovered dual-wall heat shrink tube 208 (segment) in place over a neurodiagnostic needle electrode pair subassembly 200 (shown in FIG. 2A) and placing the neurodiagnostic needle electrode pair subassembly into position in the mold base 404. FIG. 5A shows the unrecovered dual-wall heat shrink tube in transparency to highlight the placement within the base molding cavity 408 of the mold base.

A second step may include rotating the needle plate 416 downward into place against the mold base 404 to secure the needle electrodes 202 of the neurodiagnostic needle electrode pair subassembly 200 to bind the needle electrodes against movement in the needle cavity 410, as shown in FIG. 5D.

FIG. 5E illustrates that a third step may include rotating the leadwire plate 420 downward into place against the mold base 404 to secure the leadwires 204 of the neurodiagnostic needle electrode pair subassembly 200 to bind the leadwires against movement in the leadwire cavity 412.

FIG. 5F illustrates the needle plate 416 and leadwire plate 420 in transparency with the needle electrodes 202 and leadwires 204 held against movement.

FIG. 5G illustrates that a fourth step may include rotating the molding plate 418 downward to be in contact with the unrecovered dual-wall heat shrink tube 208. FIGS. 5H and 5I illustrate a cross-sectional view of the mold base 404 with the needle plate 416 hidden to show the molding plate 418 in contact with the unrecovered dual-wall heat shrink tube. FIG. 5J illustrates a close-up view of the molding plate in contact with the unrecovered dual-wall heat shrink tube in place in the base molding cavity 408 of the mold base 404.

A fifth step may include adding heat to the unrecovered dual-wall heat shrink tube 208 to begin thermal recovery of the unrecovered dual-wall heat shrink tube, as shown in FIG. 5K.

A sixth step may include further rotating the molding plate 418 to stay in contact with the unrecovered dual-wall heat shrink tube 208 as the dual-wall heat shrink tube recovers and resizes during thermal recovery to form a recovered dual-wall heat shrink tube 302, as shown in FIGS. 5L and 5M.

FIG. 5N illustrates the recovering dual-wall heat shrink tube at the point the adhesive 212 (adhesive glue) lining reaches the state of not containing air voids, at which point hydraulic forces develop in the molten adhesive with resulting flow patterns as approximated by the arrows in FIG. 5N. These hydraulic pressure forces tend to force the recovering dual-wall heat shrink outward against the inside surface of the molding cavity, ensuring that features of the neurodiagnostic needle electrode pair subassembly 200 do not yield visible flaws in the outer dimensions of the formed dual-wall heat shrink hub.

A seventh step may include continuing to rotate the molding plate 418 into fully mated position with the mold base 404 as illustrated in FIG. 5O. When designed correctly, the volume of the molding cavity will be adequate to displace and reshape the freshly recovered dual-wall heat shrink with underlying molten adhesive 212 into the desired outer shape while still allowing the molten adhesive to flow and encapsulate as much of the underlying assembly structures as possible while still forcing extrusion of the desired quantity of molten adhesive out of the openings at the ends of the dual-wall heat shrink exoskeleton, as shown in FIG. 5O.

An eighth step may include opening the molding plate 418, the leadwire plate 420, and the needle plate 416 to expose the neurodiagnostic needle electrode pair assembly 300 (with recovered dual-wall heat shrink tube 302) for removal, as shown in FIGS. 5P and 5Q.

A final step may include removing the neurodiagnostic needle electrode pair assembly 300 from the dual-wall heat shrink forming mold 400, as shown in FIG. 5R.

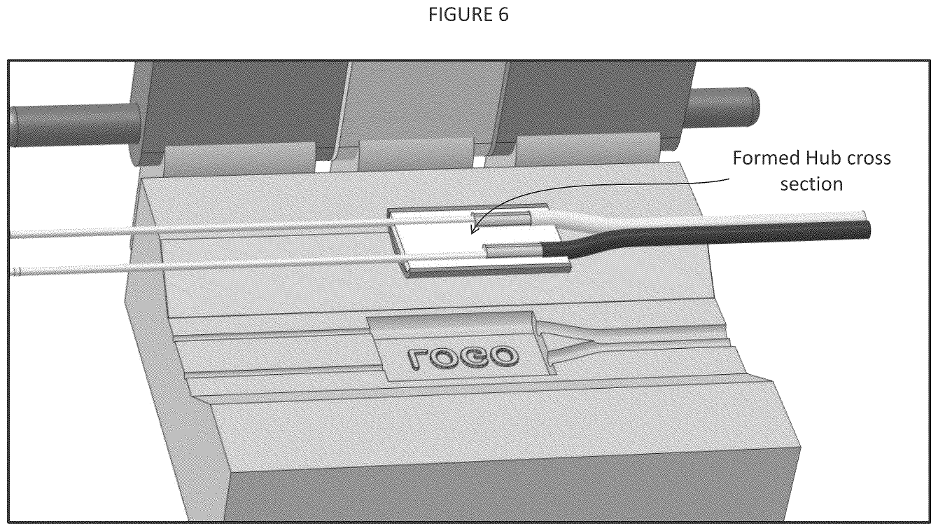

FIG. 6, a cross-sectional view of FIG. 5R, illustrates how the hydraulic forces created during the forming manufacturing process created flow of the molten adhesive 212 in such a way as to fully flow and encapsulate the underlying structures of the neurodiagnostic needle electrode pair subassembly 200.

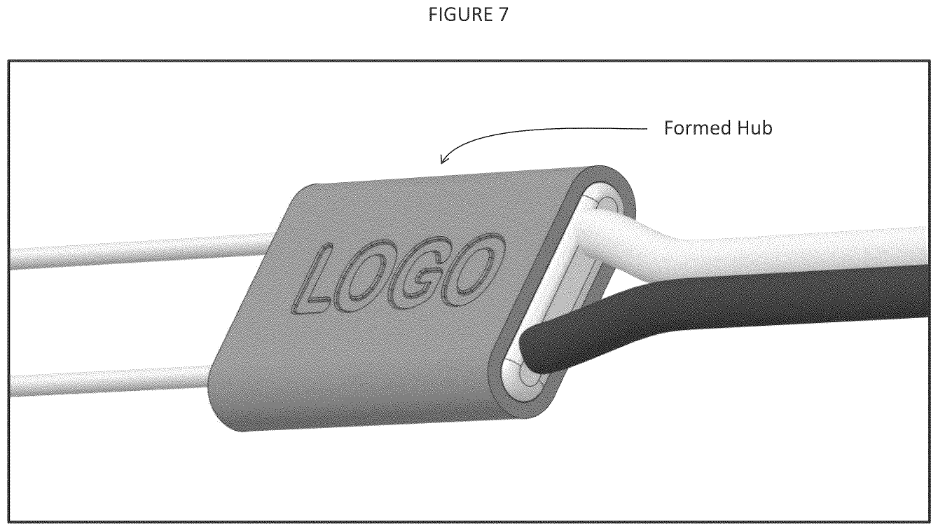

FIG. 7 illustrates a formed dual-wall heat shrink hub with consistent outer shape formed over a neurodiagnostic needle electrode pair subassembly 200 (shown in FIG. 2A) using a dual-wall heat shrink forming mold (shown in FIGS. 4A and 4B), according to some examples.

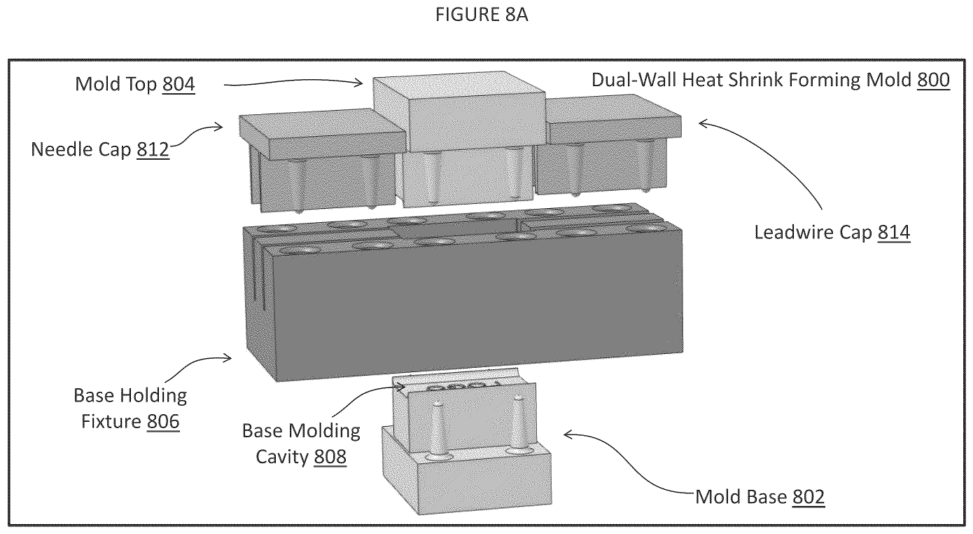

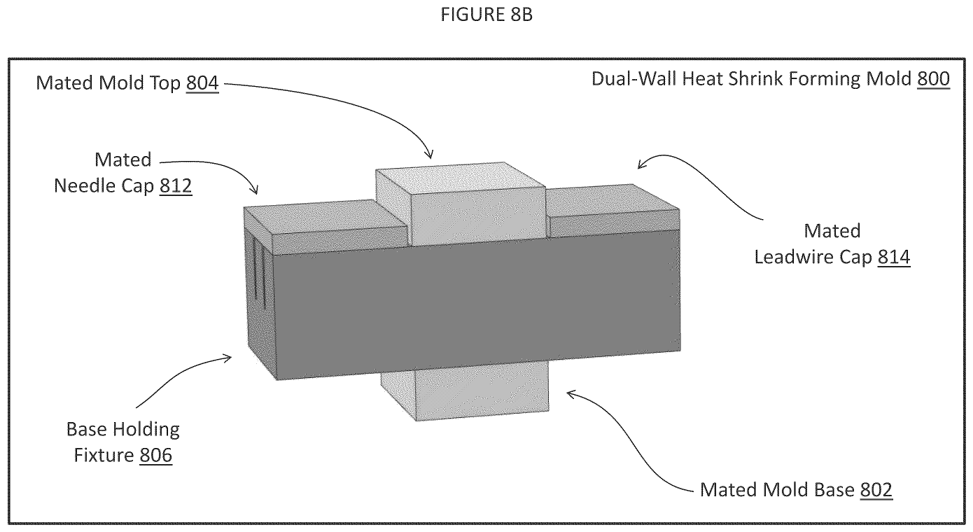

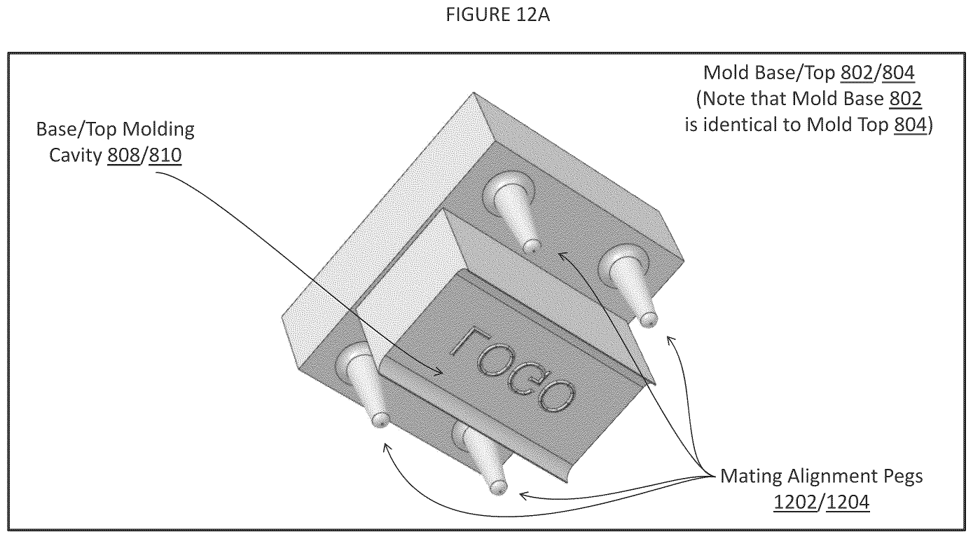

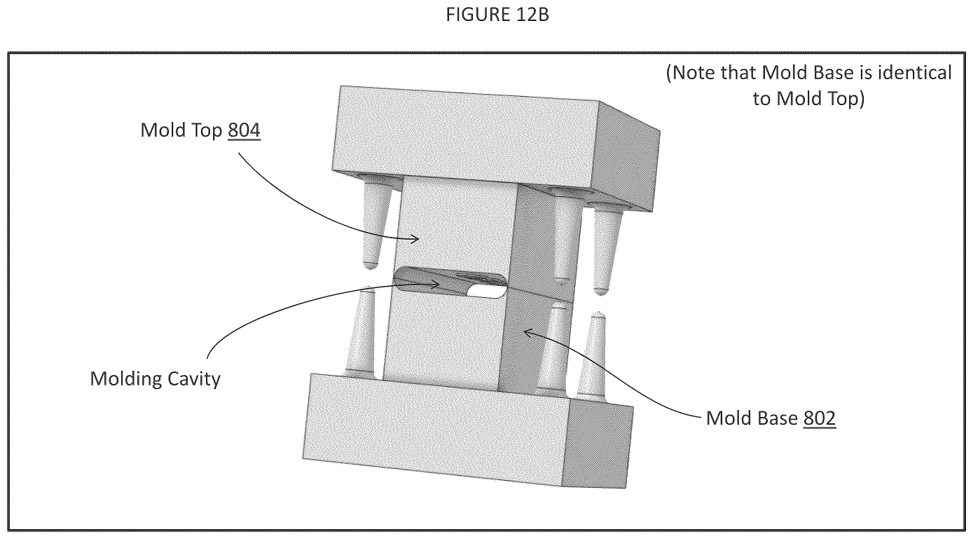

FIGS. 8A and 8B illustrate another example implementation of a dual-wall heat shrink forming mold 800 suited for producing a formed dual-wall heat shrink hub on a neurodiagnostic needle electrode pair assembly 300. Again, the neurodiagnostic needle electrode pair assembly may include a recovered dual-wall heat shrink tube 302 as an exoskeleton, and the adhesive 212 (adhesive glue) of the dual-wall heat shrink as electrical insulation and structural fill around the neurodiagnostic needle electrode pair subassembly 200. In this implementation, the dual-wall heat shrink forming mold may include a mold base 802, a mold top 804 and a base holding fixture 806. The mold base may include a base molding cavity 808, and the mold top may include a top molding cavity 810 (shown in FIG. 12A); and when mated the base molding cavity and the top molding cavity may form a molding cavity (also referred to as a forming mold cavity). As also shown, the dual-wall heat shrink forming mold may also include a needle cap 812 and/or a leadwire cap 814.

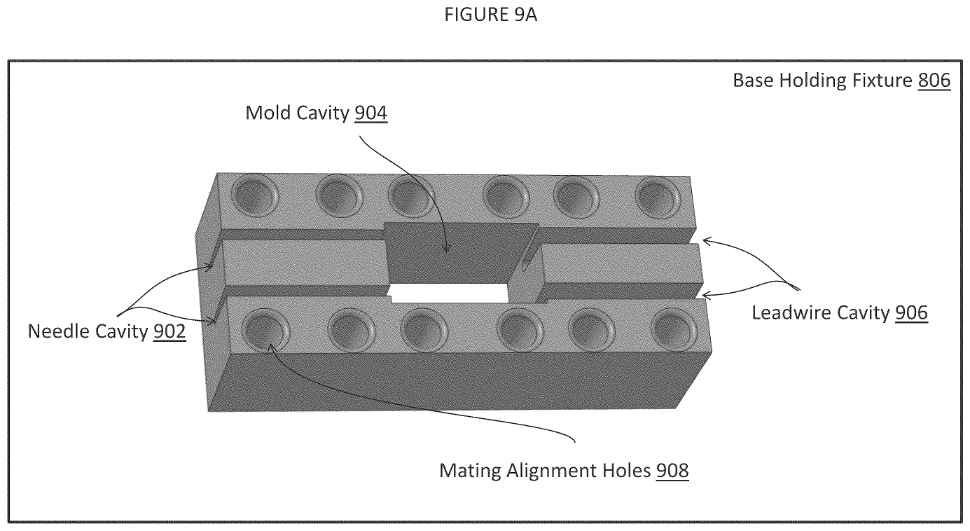

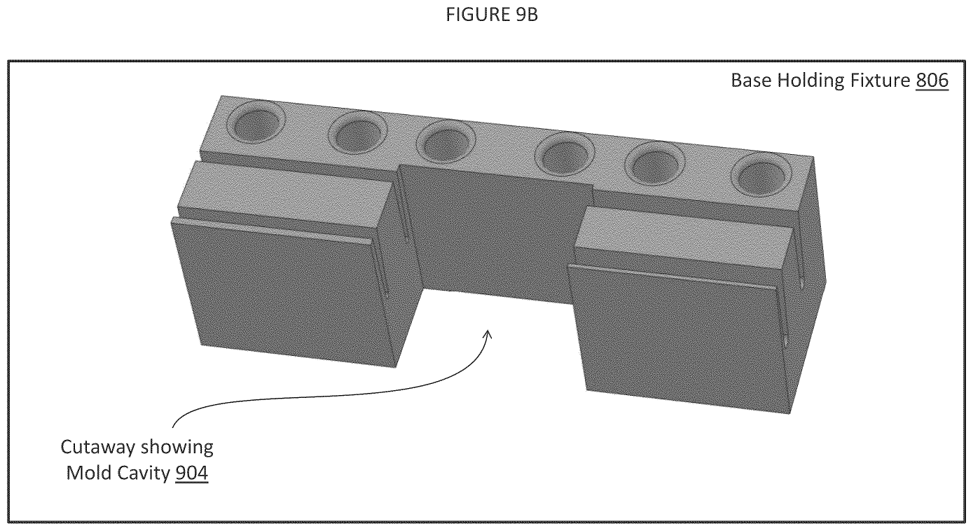

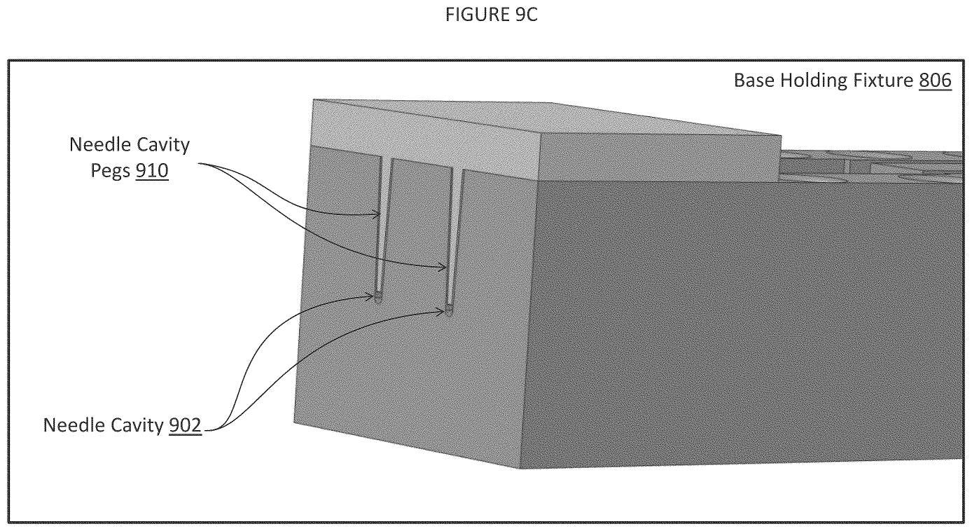

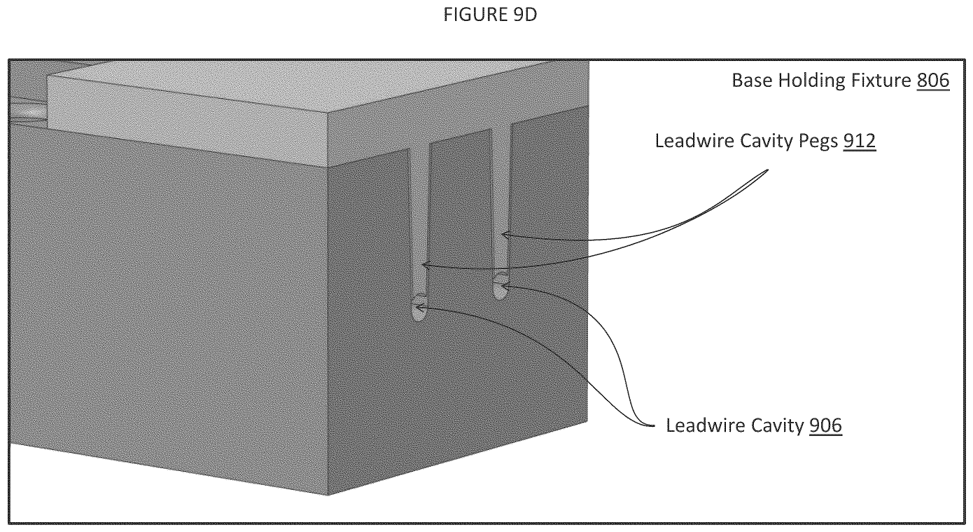

FIGS. 9A, 9B and 9C illustrate multiple views of the base holding fixture 806. This example implementation of the base holding fixture includes needle cavities 902, a mold cavity 904, and leadwire cavities 906. The base holding fixture may also include mating alignment holes 908 which ensure aligned fit when the needle cap 812, the leadwire cap 814, the mold top 804, and the mold base 802 are mated with the base holding fixture. This example implementation of the needle cap includes needle cavity pegs 910, and the leadwire cap includes leadwire cavity pegs 912.

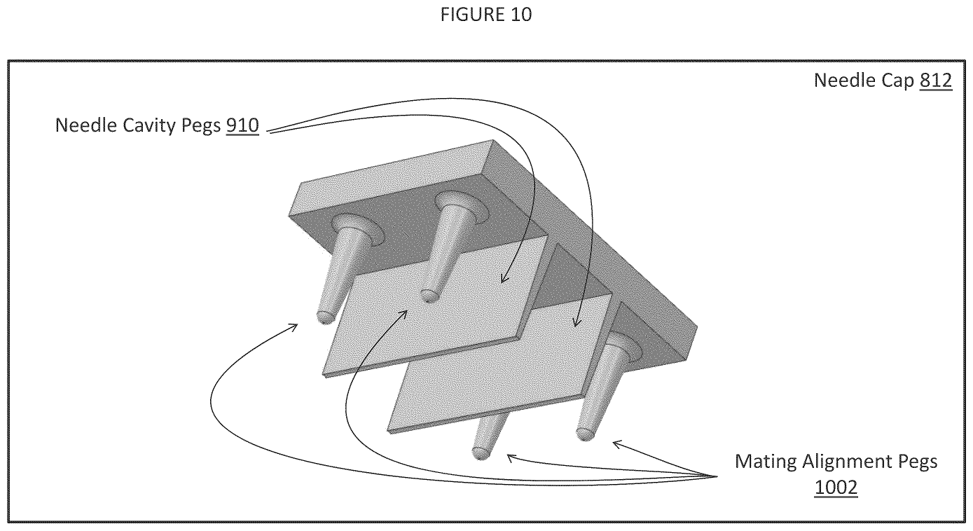

FIG. 10 illustrates the needle cap 812. The needle cap may mate with the base holding fixture 806 to tightly bind the needle electrodes 202 against movement in the needle cavity 902. This example implementation of the needle cap further includes mating alignment pegs 1002.

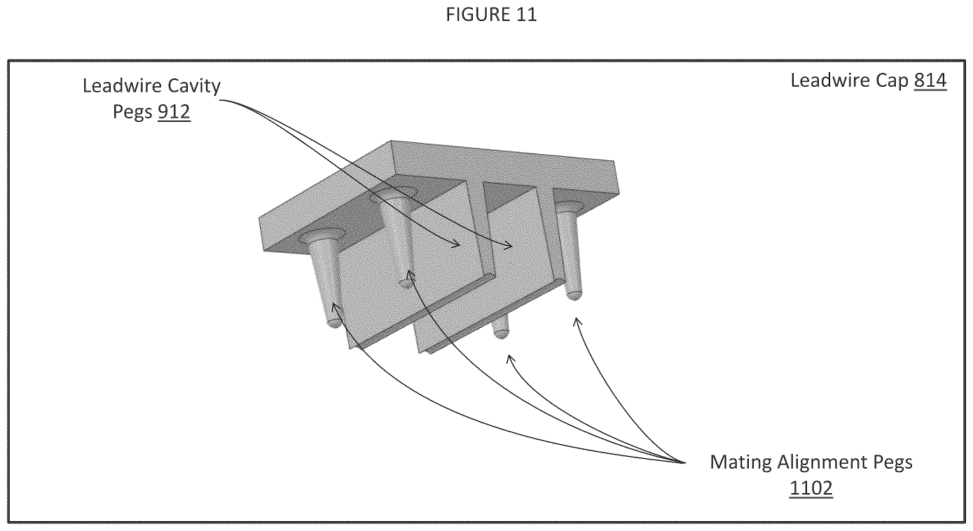

FIG. 11 illustrates the leadwire cap 814. The leadwire cap may mate with the base holding fixture 806 to tightly bind the leadwires 204 against movement in the leadwire cavity 906. This example implementation of the leadwire cap further includes mating alignment pegs 1102.

FIGS. 12A and 12B illustrate multiple views of the mold top 804 and mold base 802 which may comprise identical structures. The top molding cavity 810 of the mold top and the base molding cavity 808 of the mold base may mate to form a molding cavity within the mold cavity 904 of the base holding fixture 806. This example implementation of the mold top/mold base further includes mating alignment pegs 1202/1204.

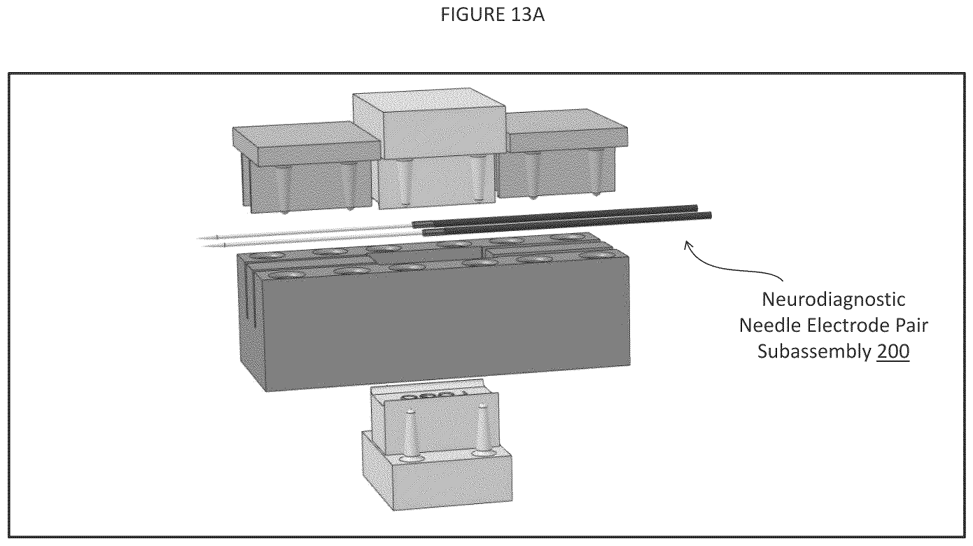

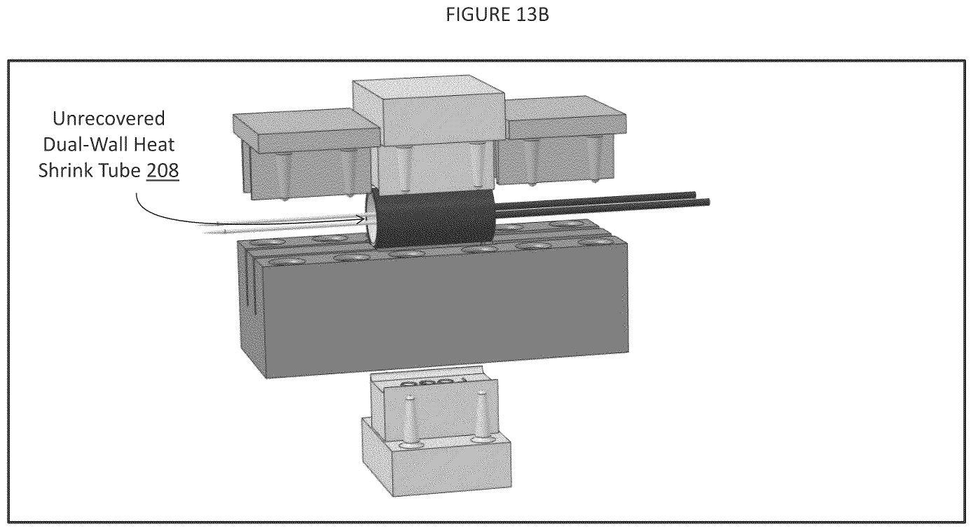

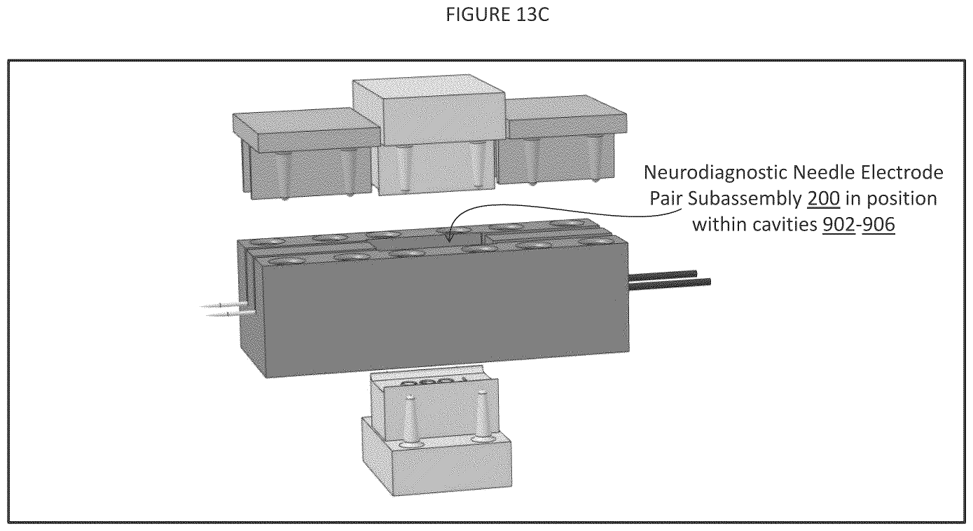

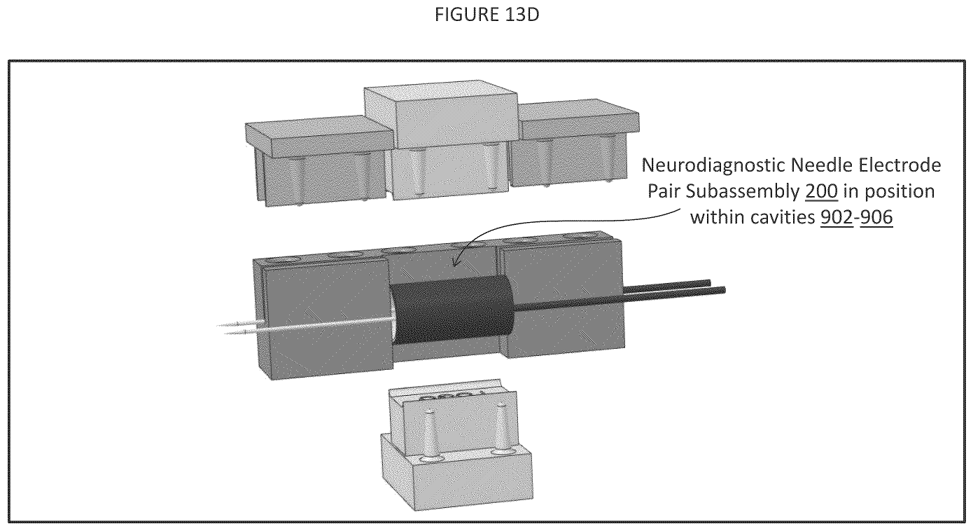

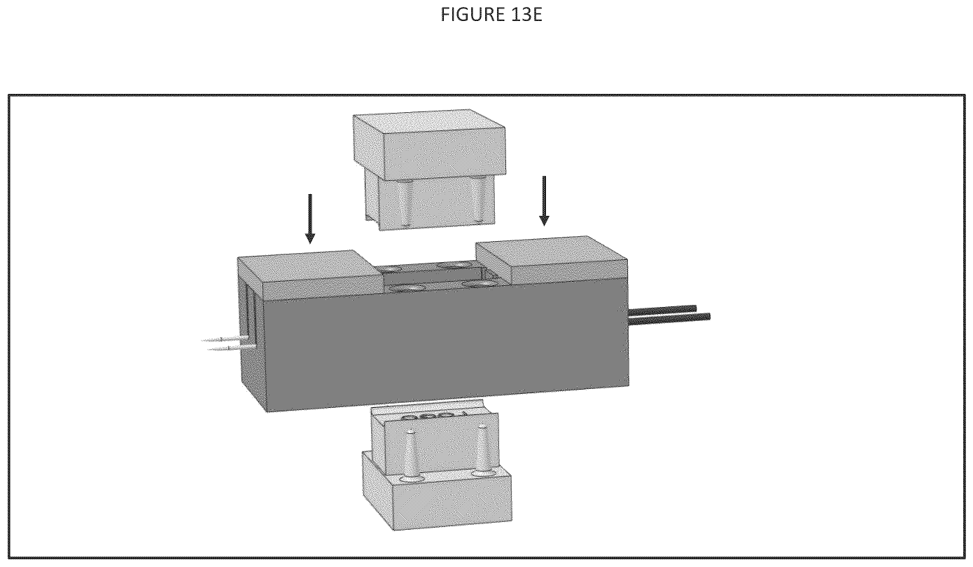

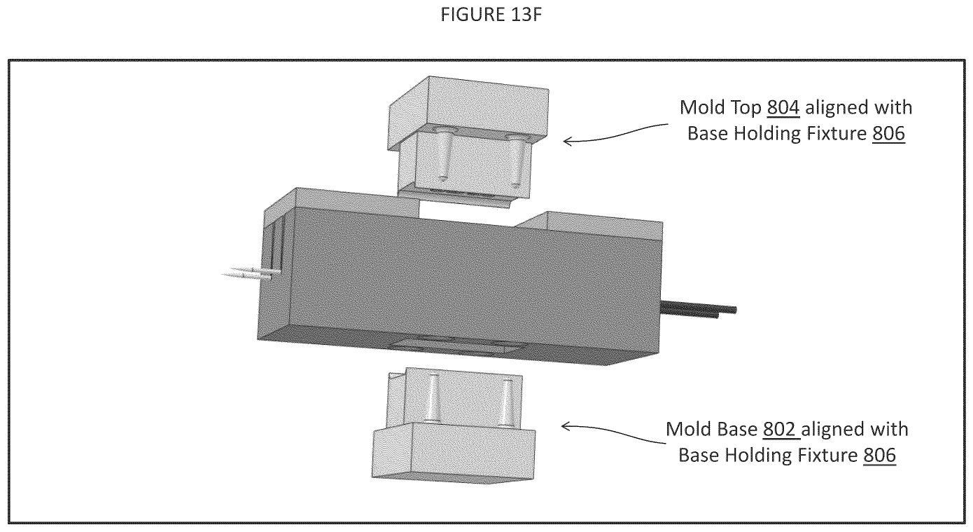

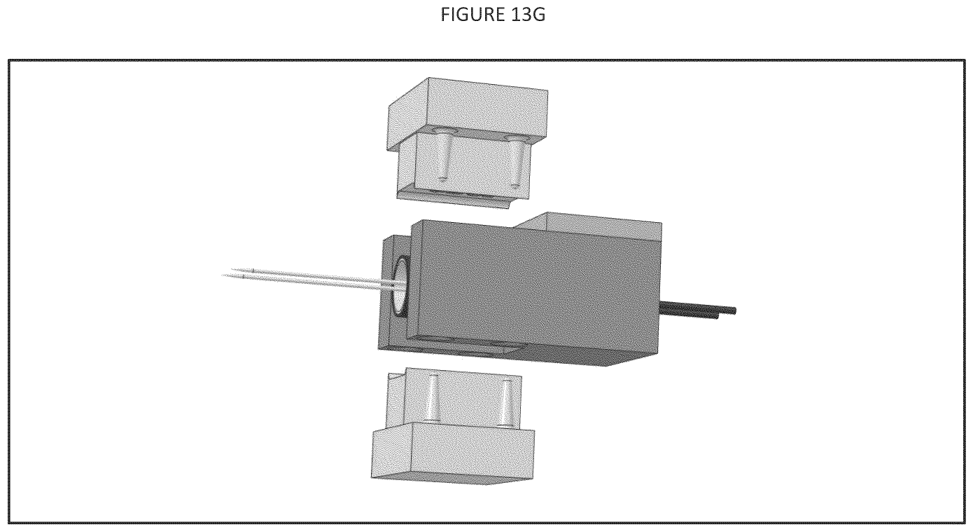

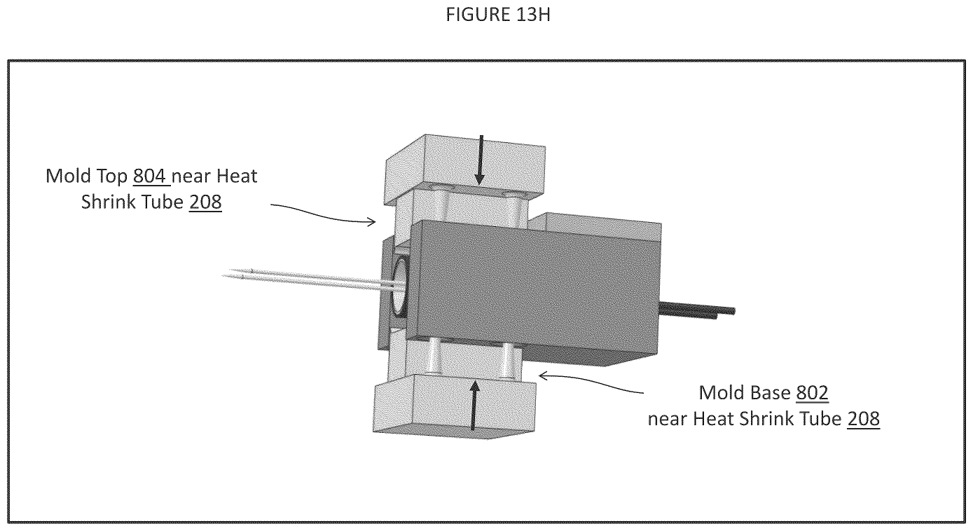

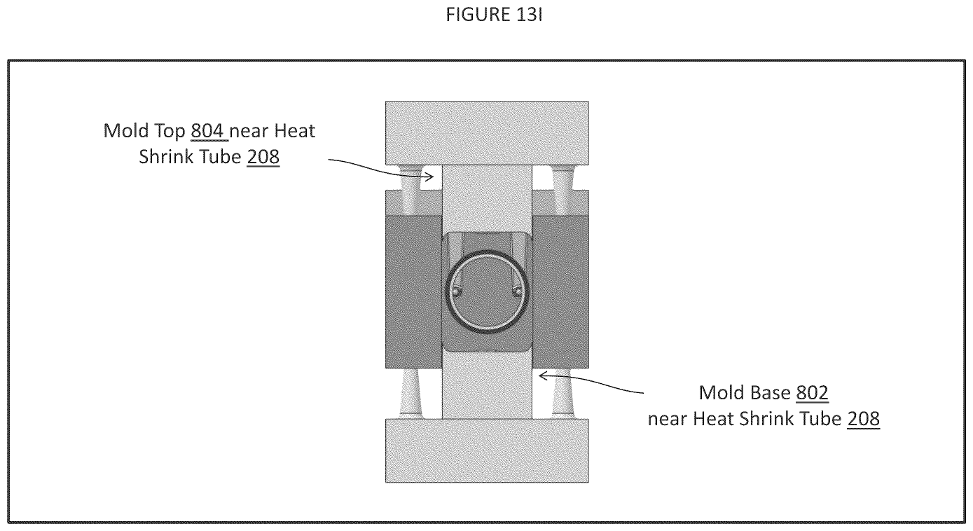

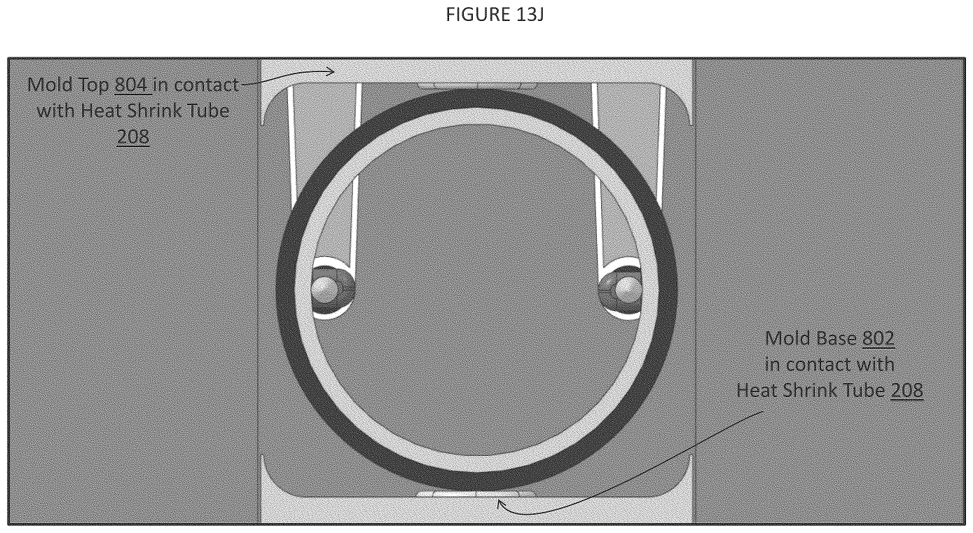

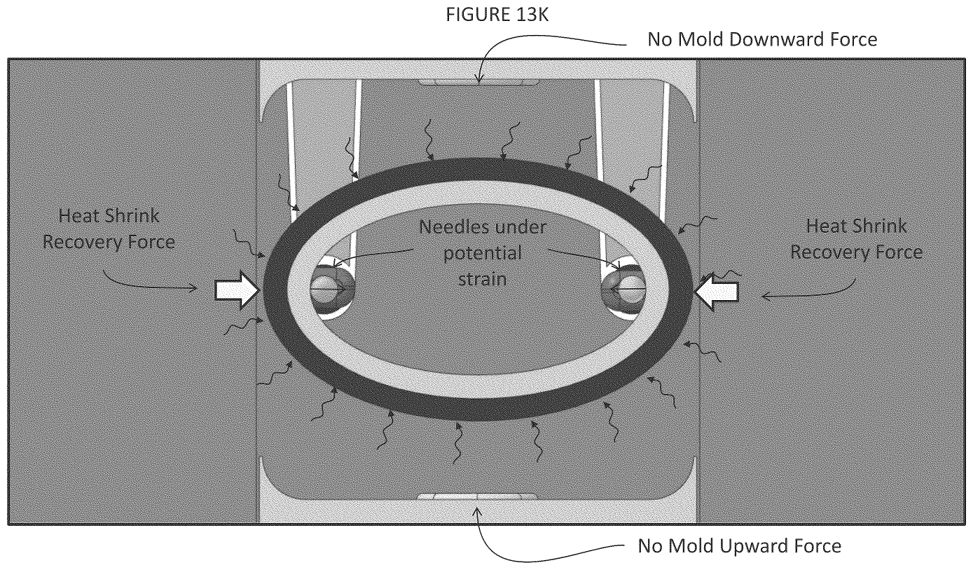

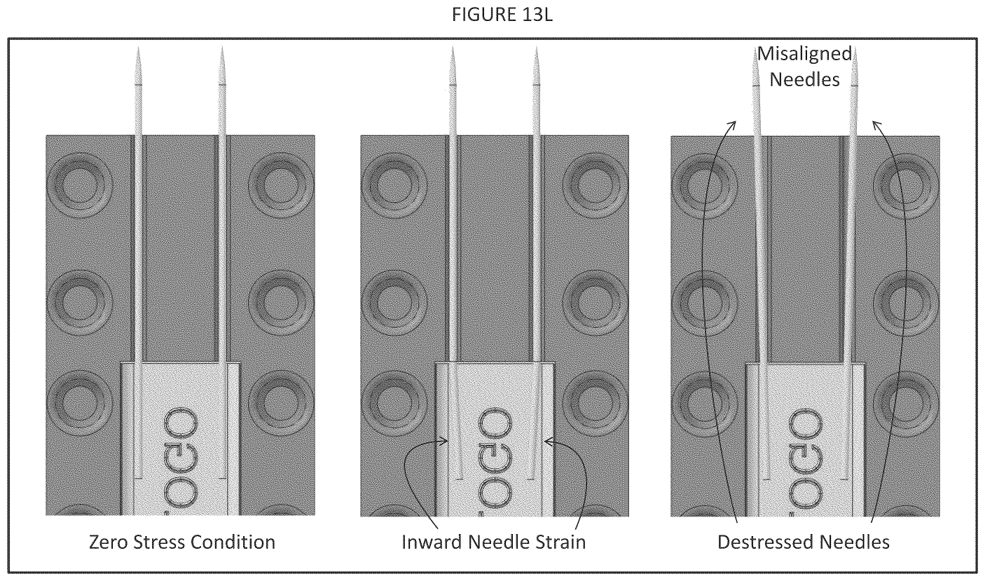

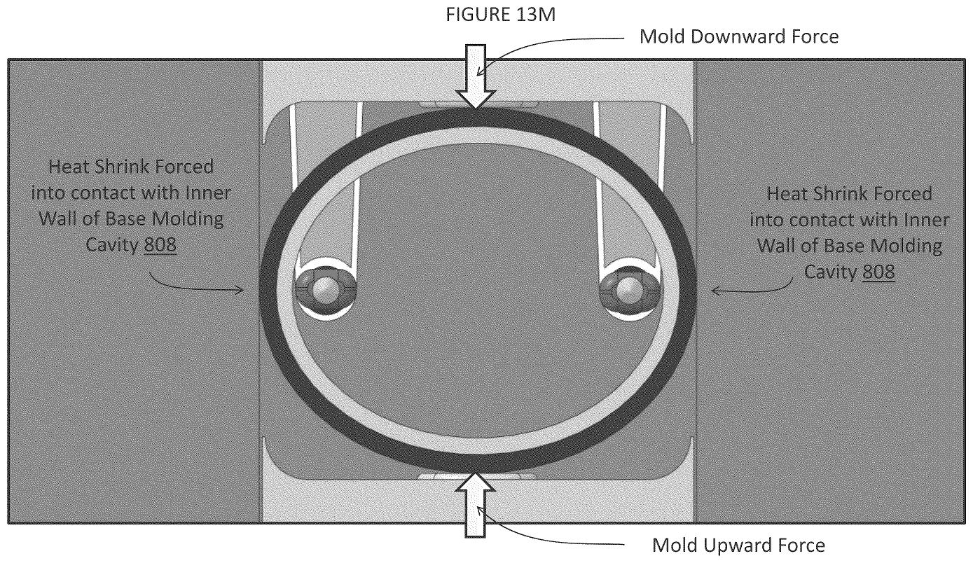

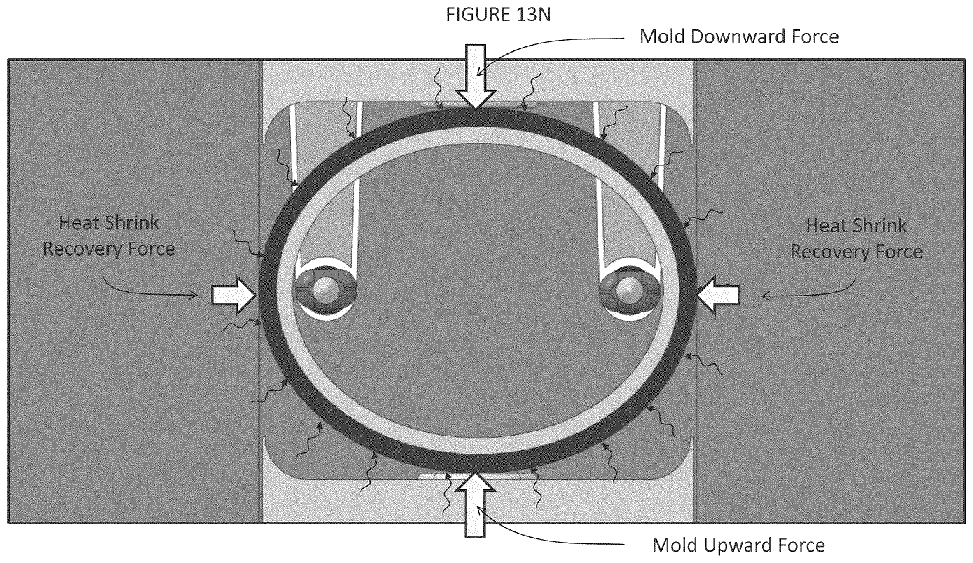

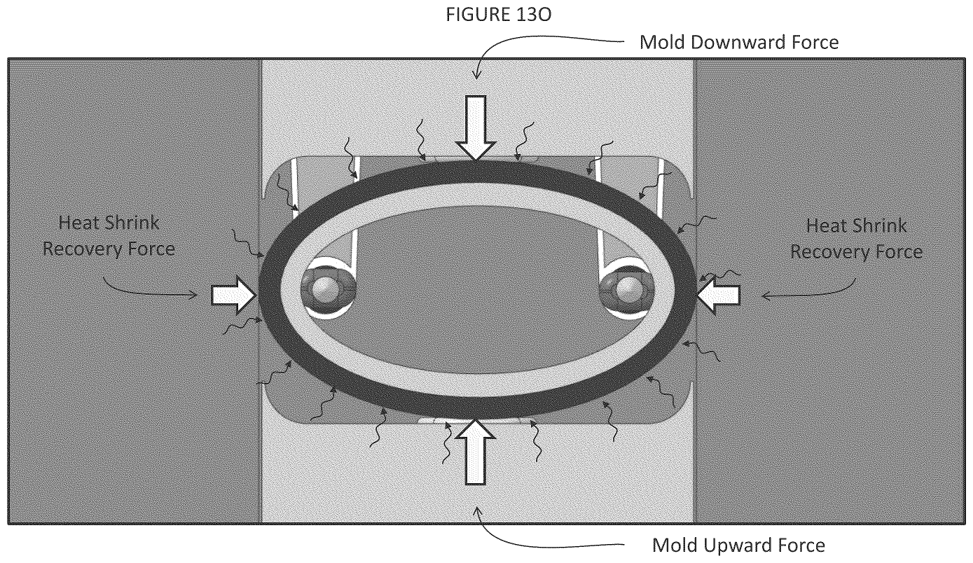

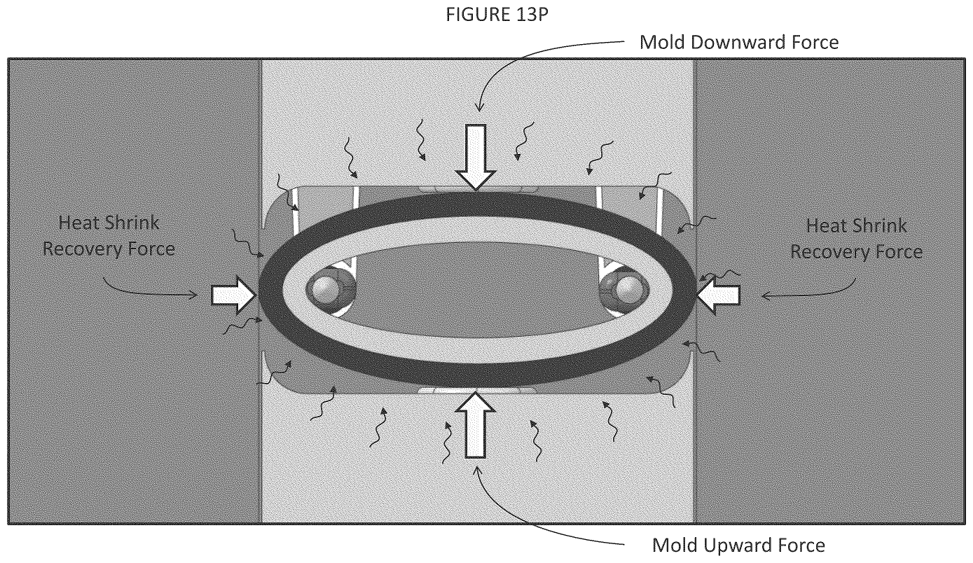

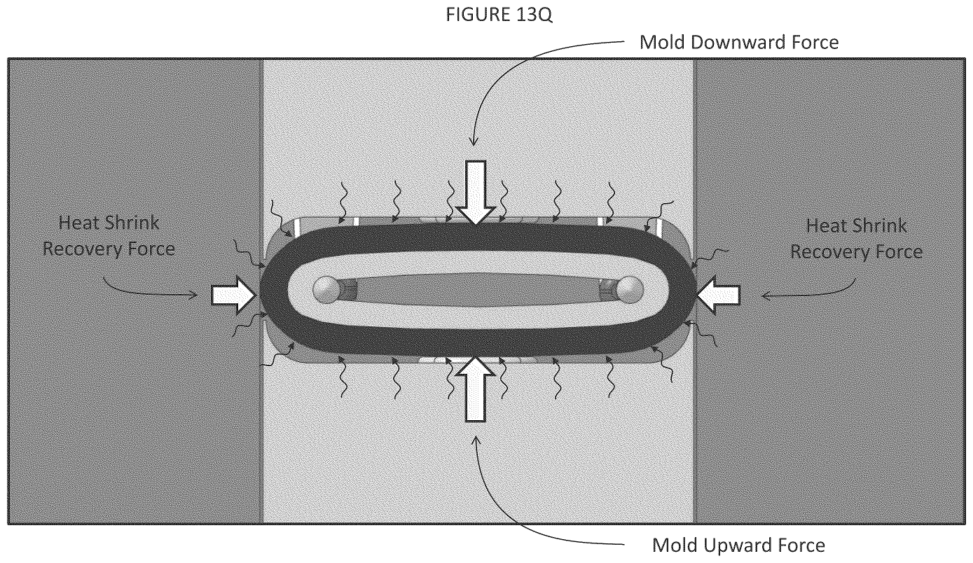

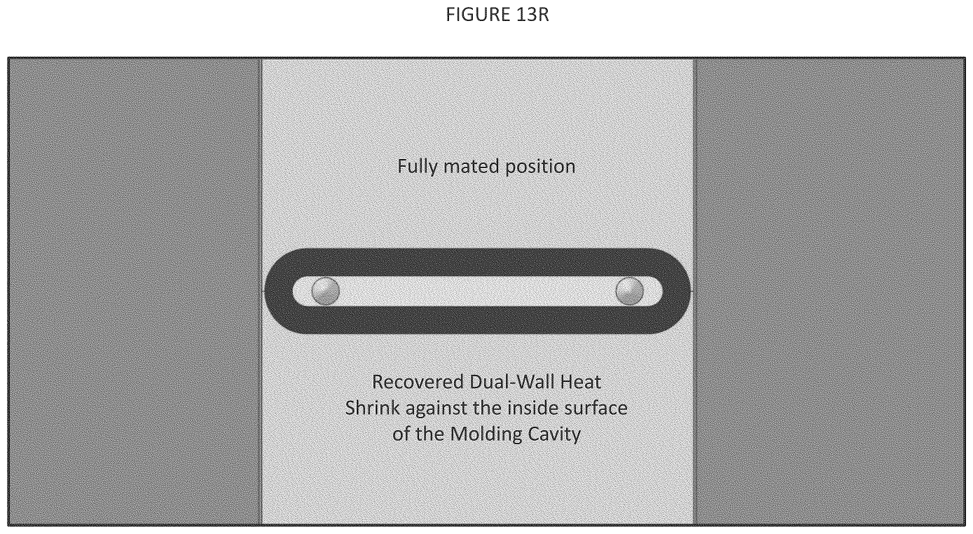

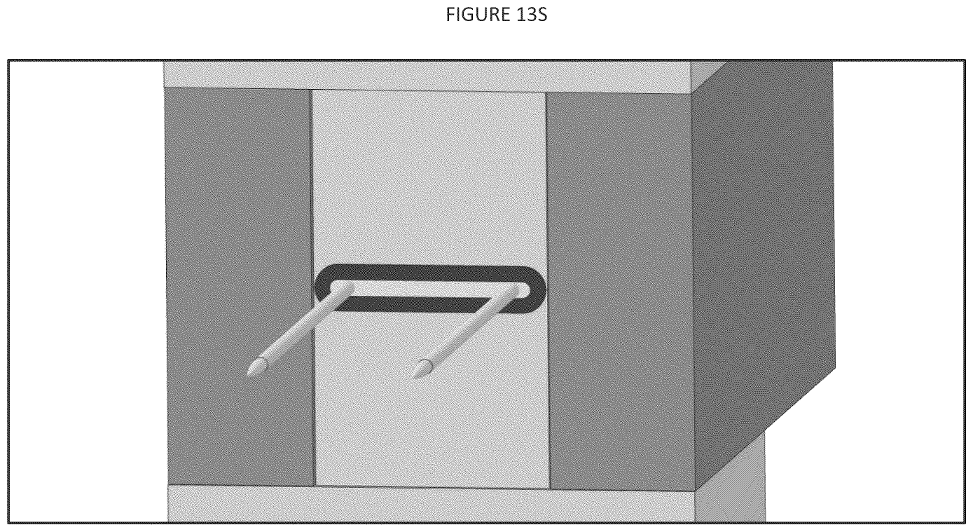

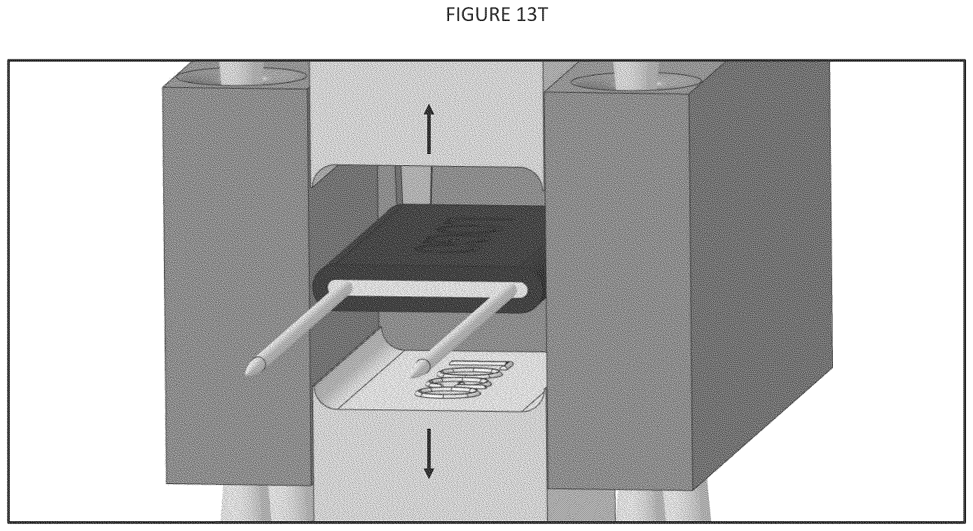

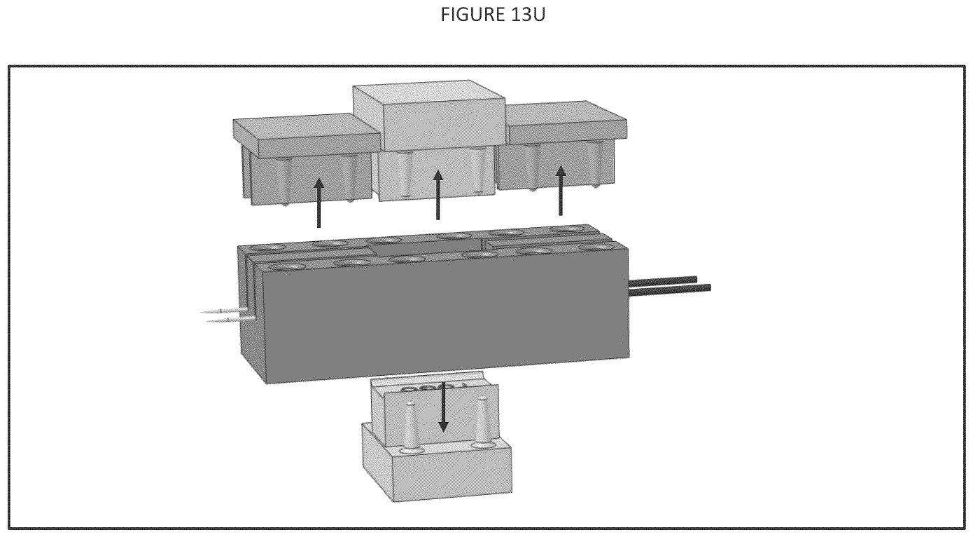

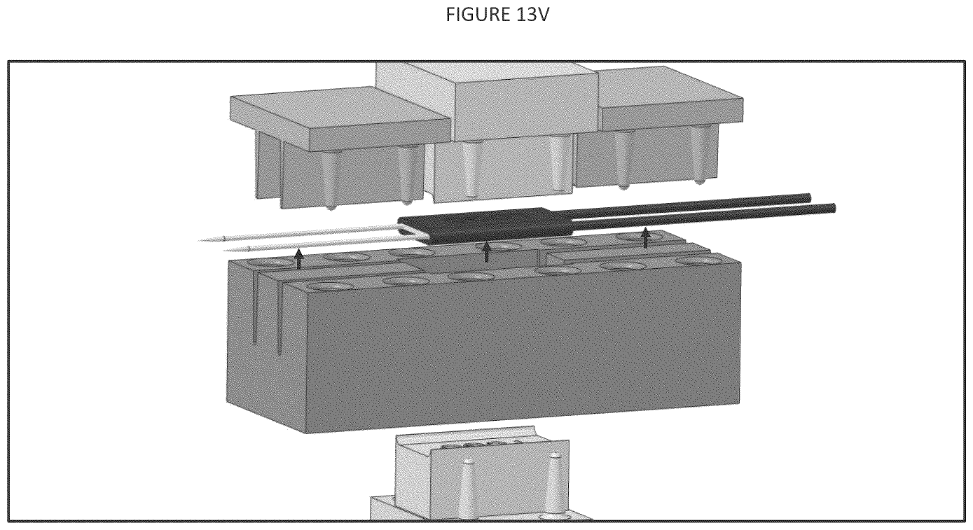

According to other examples, FIGS. 13A, 13B, 13C, 13D, 13E, 13F, 13G, 13H, 13I, 13J, 13K, 13L, 13M, 13N, 130, 13P, 13Q, 13R, 13S, 13T, 13U and 13V show steps of a method of manufacturing a formed dual-wall heat shrink hub on a neurodiagnostic needle electrode pair utilizing an implementation of a dual-wall heat shrink forming mold 800 (as shown in FIGS. 8A and 8B).

A first step may include placing a neurodiagnostic needle electrode pair subassembly 200 (as shown in FIG. 2A) and an unrecovered dual-wall heat shrink tube 208 into position within the cavities 902-906 of the base holding fixture 806, as shown in FIGS. 13A, 13B, and 13C. FIG. 13D is a cross-sectional view of FIG. 13C to illustrate a neurodiagnostic needle electrode pair subassembly in position within the cavities of the base holding fixture.

A second step may include placing the needle cap 812 and the leadwire cap 814 into fully mated positions with the base holding fixture 806 to secure the needle electrodes 202 and the leadwires 204 of the neurodiagnostic needle electrode pair subassembly 200 against movement in the needle cavity 902 and the leadwire cavity 906, as shown in FIG. 13E.

FIGS. 13F, 13G, 13H, 13I, and 13J illustrate that a third step may include bringing the mold top 804 and mold base 802 into proper alignment with the base holding fixture 806 and moving them to be in contact with the unrecovered dual-wall heat shrink tube 208. FIGS. 13G, 13H, and 13I illustrate cross-sectional views of the base holding fixture to show placement of the mold top and mold base in near contact with the unrecovered dual-wall heat shrink tube. FIG. 13J illustrates a close-up view of the base holding fixture with the mold top and mold base in contact with the unrecovered dual-wall heat shrink tube.

FIG. 13K illustrates what may occur if heat is added to begin thermal recovery of the dual-wall heat shrink tube 208 without the presence of mold forces created by moving the mold top 804 downward and the mold base 802 upward against the recovering dual-wall heat shrink tube. The recovering dual-wall heat shrink after coming in contact with the needle electrodes 202 may create recovery forces large enough to elastically strain the needle electrodes inside of the recovering dual-wall heat shrink tube. The effects of this elastic needle strain may result in misaligned needle electrodes when the neurodiagnostic needle electrode pair is removed from the constraints of the needle cavity 902 of the base holding fixture 806, as shown in FIG. 13L. The next steps of manufacture are designed to minimize the opportunity for misaligned needle electrodes as a result of heat shrink recovery forces stressing the needle electrodes during the thermal recovery process.

A fourth step may include moving the mold top 804 and mold base 802 into closer contact with the unrecovered dual-wall heat shrink tube 208 in such a way as to force the sides of the unrecovered dual-wall heat shrink tube into contact with the inner wall of the mold cavity 904 of the base holding fixture 806, as shown in FIG. 13M.

A fifth step may include adding heat to begin thermal recovery of the unrecovered dual-wall heat shrink tube 208, as shown in FIG. 13N. As heat is applied, the mold top 804 and mold base 802 are moved sequentially closer to full mating position so that the downward and upward mold forces created by this movement are continually larger than the heat shrink recovery forces, as shown in FIGS. 13N, 130, 13P, and 13Q. FIG. 13R illustrates the point at which the mold top and mold base are in fully mated position and heat and applied force may be removed. When designed correctly, the volume of the molding cavity will be adequate to displace and shape the freshly recovered dual-wall heat shrink with underlying molten adhesive 212 into the desired outer shape while still allowing the molten adhesive to flow and completely encapsulate the underlying assembly structures. FIG. 13S illustrates an angled view of FIG. 13R.

A sixth step may include separating the mold top 804 and the mold base 802 from the base holding fixture 806 to expose the neurodiagnostic needle electrode pair assembly 300 (with recovered dual-wall heat shrink tube 302) for removal, as shown in FIG. 13T.

A seventh step may include removing the leadwire cap 814, the needle cap 812, the mold top 804, and the mold base 802 from the base holding fixture 806 to expose the formed neurodiagnostic needle electrode pair for removal, as shown in FIG. 13U.

A final step may include removing the neurodiagnostic needle electrode pair assembly 300 from the base holding fixture 806 of the dual-wall heat shrink forming mold 800, as shown in FIG. 13V.

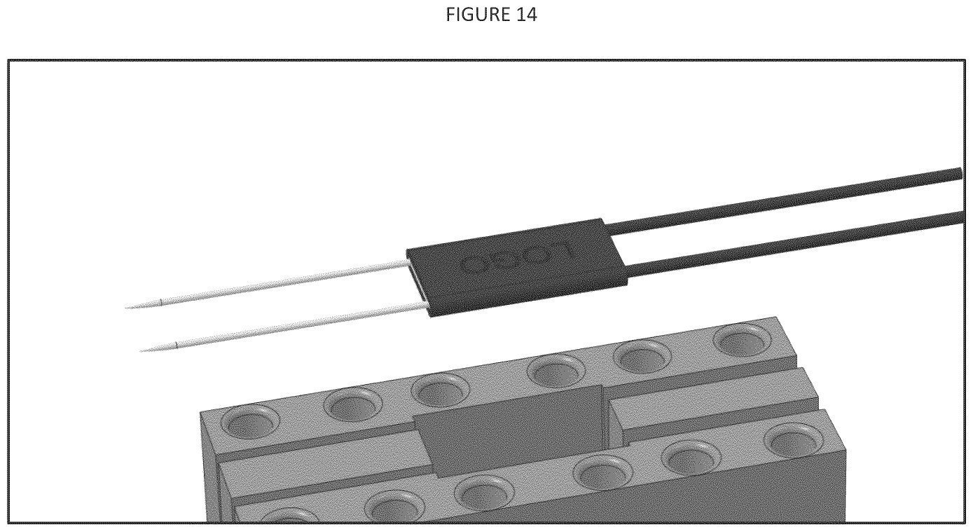

FIG. 14 illustrates the neurodiagnostic needle electrode pair assembly 300 including a formed dual-wall heat shrink hub with consistent outer shape formed over a neurodiagnostic needle electrode pair subassembly 200 that includes a needle electrode pair 202, a leadwire pair 204, and a securement such as electrical connections 206 performing electrical and mechanical connection of the needle electrodes to the leadwires (shown in FIG. 2A), according to some examples.

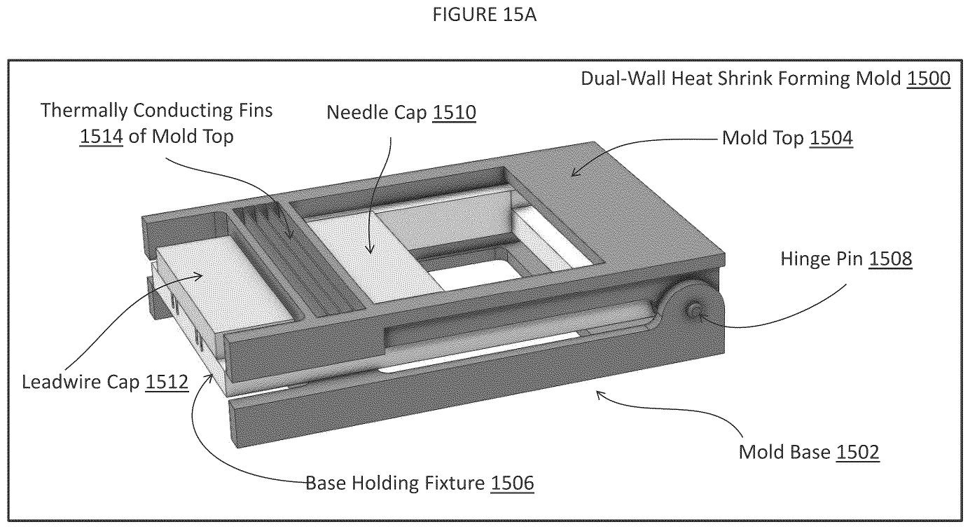

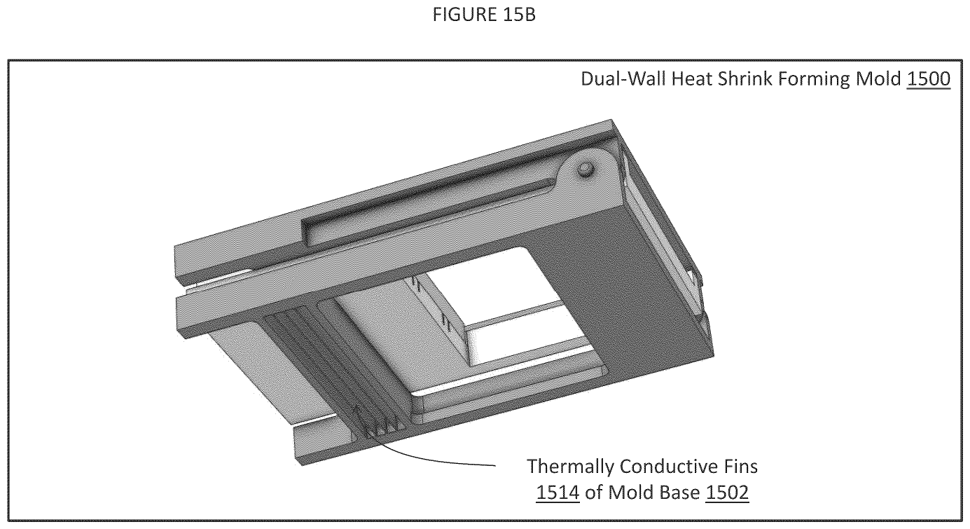

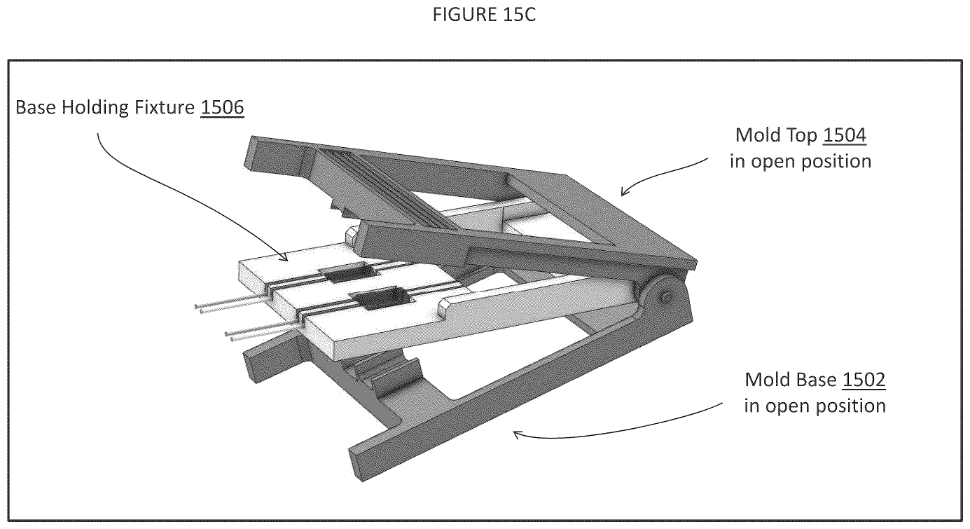

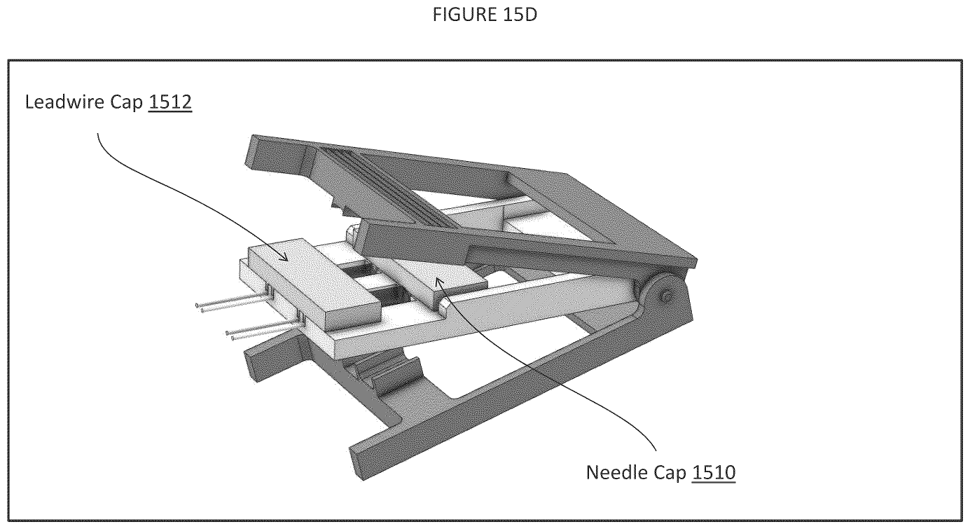

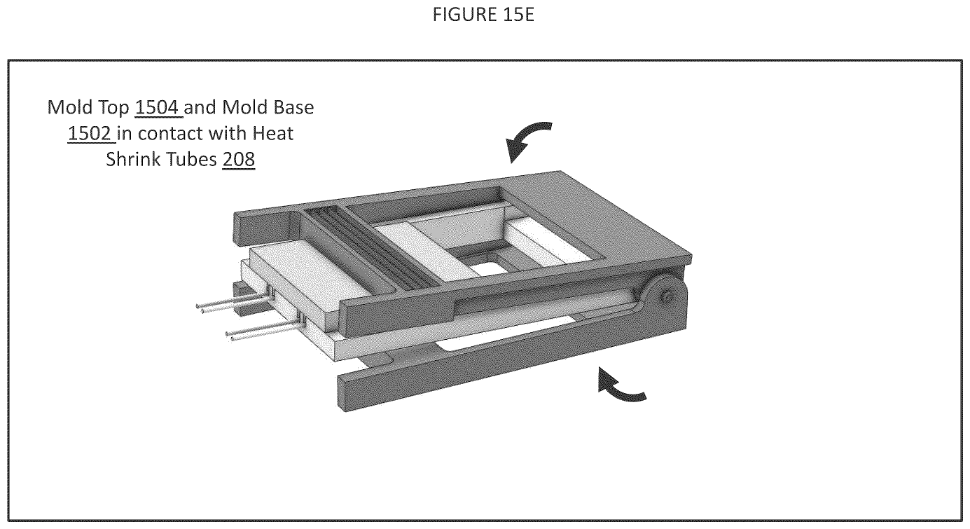

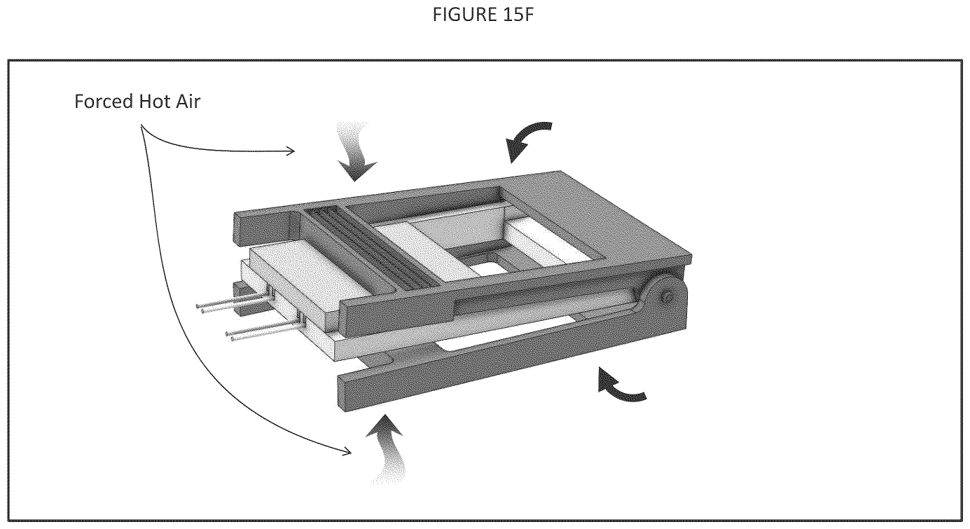

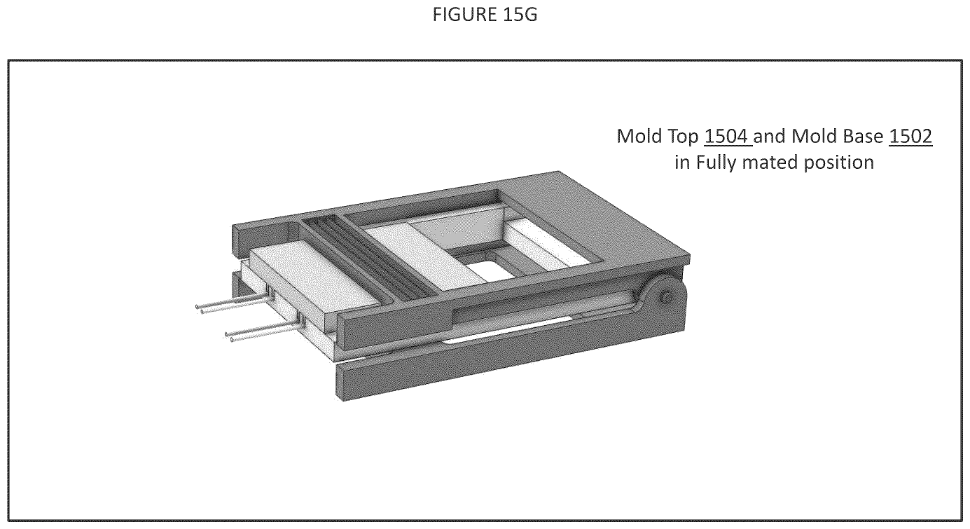

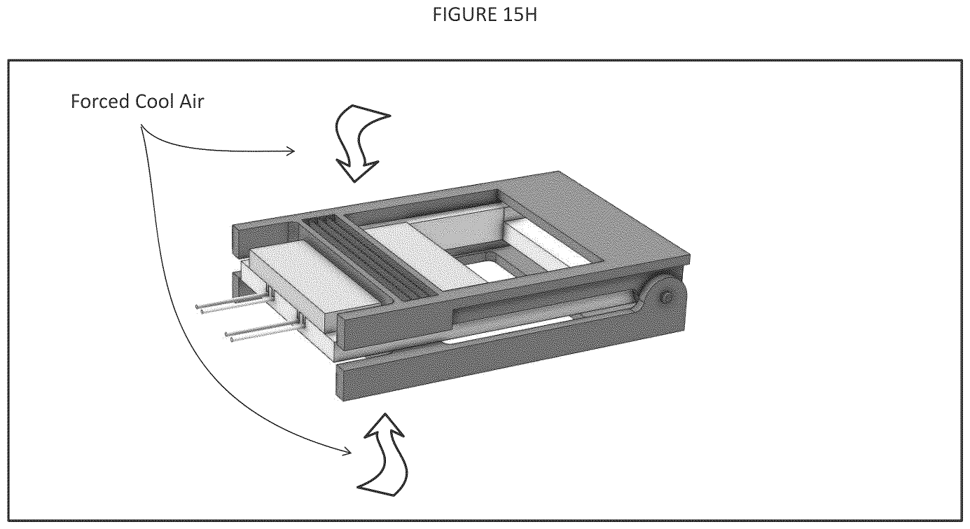

FIGS. 15A and 15B illustrate another example implementation of a dual-wall heat shrink forming mold 1500 suited for producing a formed dual-wall heat shrink hub on a neurodiagnostic needle electrode pair assembly 300. Again, the neurodiagnostic needle electrode pair assembly may include a recovered dual-wall heat shrink tube 302 as an exoskeleton, and the adhesive 212 (adhesive glue) of the dual-wall heat shrink as electrical insulation and structural fill around the neurodiagnostic needle electrode pair subassembly 200.

As shown, in this implementation, the dual-wall heat shrink forming mold 1500 may include a mold base 1502, a mold top 1504 and a base holding fixture 1506. The mold base and the mold top may be connected by a hinge pin 1508. Although not separately shown, the mold base may include a base molding cavity, and the mold top may include a top molding cavity; and when mated the base molding cavity and the top molding cavity may form a molding cavity (also referred to as a forming mold cavity). The base holding fixture may also include cavities, such as needle cavities, mold cavity and/or leadwire cavities. The dual-wall heat shrink forming mold may also include a needle cap 1510 and/or a leadwire cap 1512. And as also shown, either or both of the mold base or mold top may include thermally conducting fins 1514.

FIG. 15C shows neurodiagnostic needle electrode pair subassemblies 200 (as shown in FIG. 2A) and unrecovered dual-wall heat shrink tubes 208 in position within the cavities of the base holding fixture 1506 with the mold top 1504 and the mold base 1502 in open position.

FIG. 15D illustrates the needle cap 1510 and leadwire cap 1512 in fully mated position with the needle electrodes 202 and leadwires 204 held against movement.

FIG. 15E illustrates bringing the mold top 1504 and mold base 1502 into proper alignment with the base holding fixture 1506 and moving them to be in contact with the unrecovered dual-wall heat shrink tubes 208 in such a way as to force the sides of the unrecovered dual-wall heat shrink tubes into contact with the inner wall of cavities in the base holding fixture.

FIG. 15F illustrates adding heat to begin thermal recovery of the unrecovered dual-wall heat shrink tubes 208. Forced hot air applied against the thermally conductive fins 1514 of the mold top 1504 and/or the mold base 1502 facilitate heating of the mold cavities. As heat is applied, the mold top 1504 and mold base 1502 are moved sequentially closer to full mating position so that the downward and upward mold forces created by this movement are continually larger than the heat shrink recovery forces.

FIG. 15G illustrates the point at which the mold top 1504 and mold base 1502 are in fully mated position and heat and applied force may be removed. When designed correctly, the volume of the molding cavities will be adequate to displace and shape the freshly recovered dual-wall heat shrink with underlying molten adhesive 212 into the desired outer shape while still allowing the molten adhesive 212 to flow and completely encapsulate the underlying assembly structures.

FIG. 15H illustrates applying forced cool air against the thermally conductive fins 1514 of the mold top 1504 and/or the mold base 1502 to facilitate cooling of the entire dual-wall heat shrink forming mold 1500.

FIG. 15I illustrates removal of the formed neurodiagnostic needle electrode pair assemblies 300 from the base holding fixture 1506 of the dual-wall heat shrink forming mold 1500.

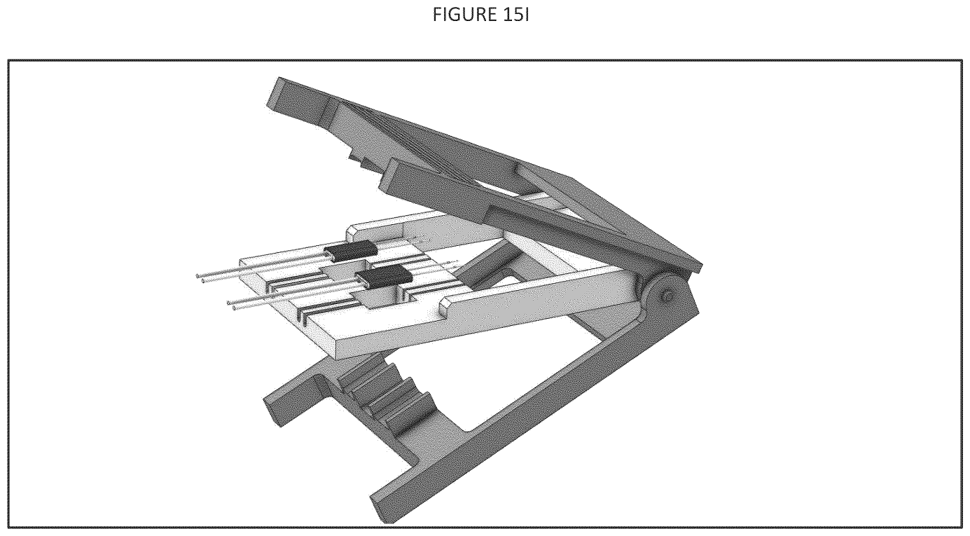

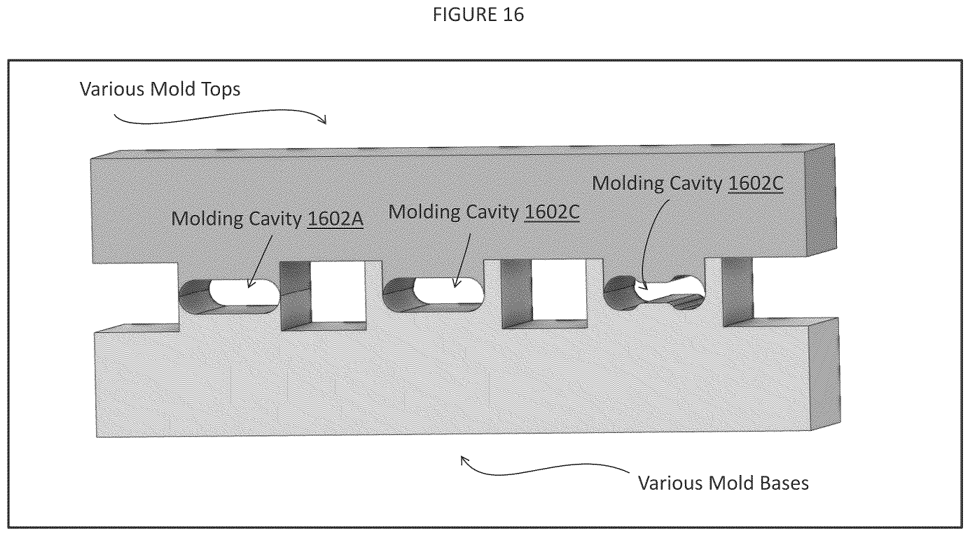

FIG. 16 illustrates various molding cavities 1602A, 1602B, 1602C that may be created when mold tops and mold bases are in fully mated position. When designed correctly, the volume of the molding cavities will be adequate to displace and shape the freshly recovered dual-wall heat shrink with underlying molten adhesive 212 into the desired outer shape while still allowing the molten adhesive to flow and completely encapsulate the underlying assembly structures. The molding cavities shown in FIG. 16 in no way represent all configurations of molding cavities but serve as an example of various types of molding cavities and are not meant to limit implementations of the present disclosure. Rather, they illustrate a few example implementations of the many types of molding cavities and are not limited to the use of these exact types or configurations. It should be noted that implementations may or may not always use the features illustrated in FIG. 16.

FIG. 16 illustrates one implementation of a molding cavity 1602A that may be created when a mold top and mold base are in a fully mated position within a base holding fixture, as shown in FIG. 13R. Also shown is an implementation of a molding cavity 1602B that includes cavity sidewalls as part of the mold base. And another implementation of a molding cavity 1602C includes an indentation feature which is designed to minimize the opportunity for misaligned needle electrodes 202 as a result of heat shrink recovery forces stressing the needle electrodes during the thermal recovery process.

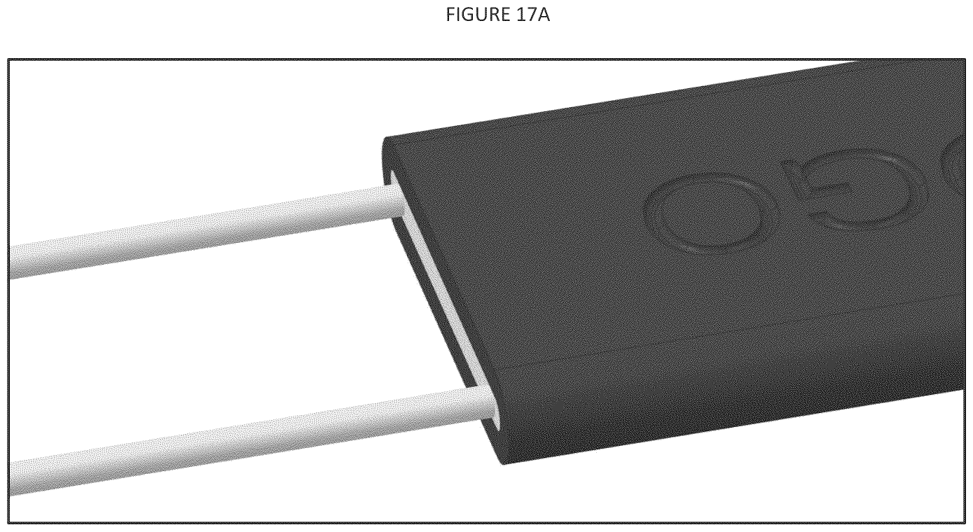

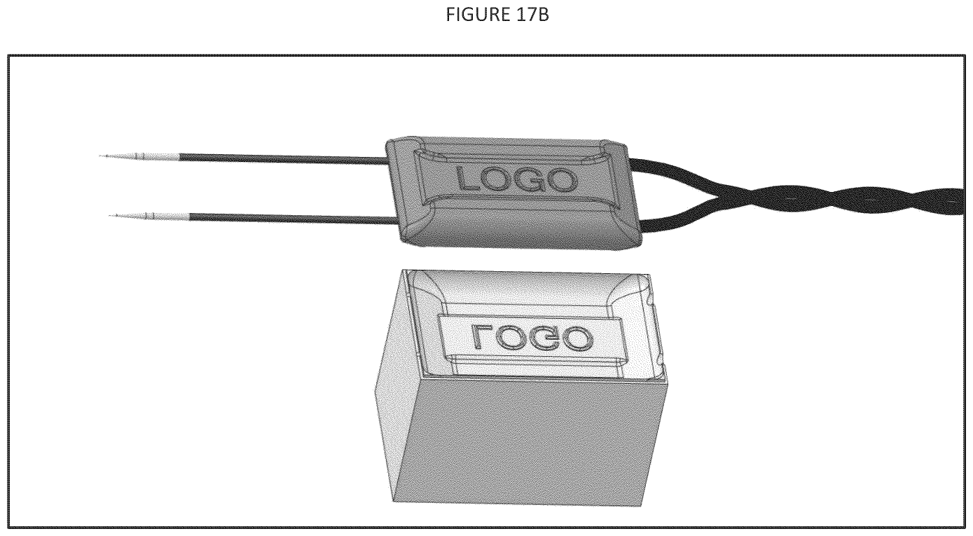

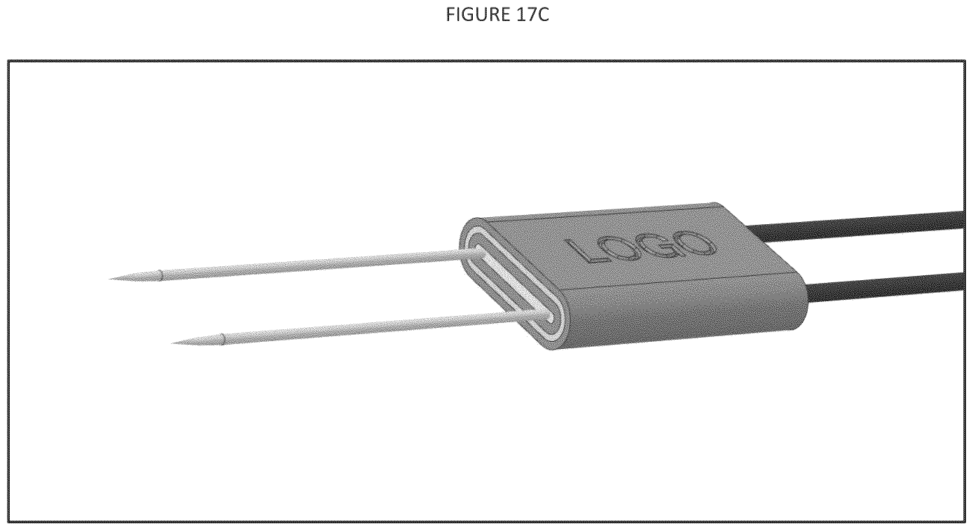

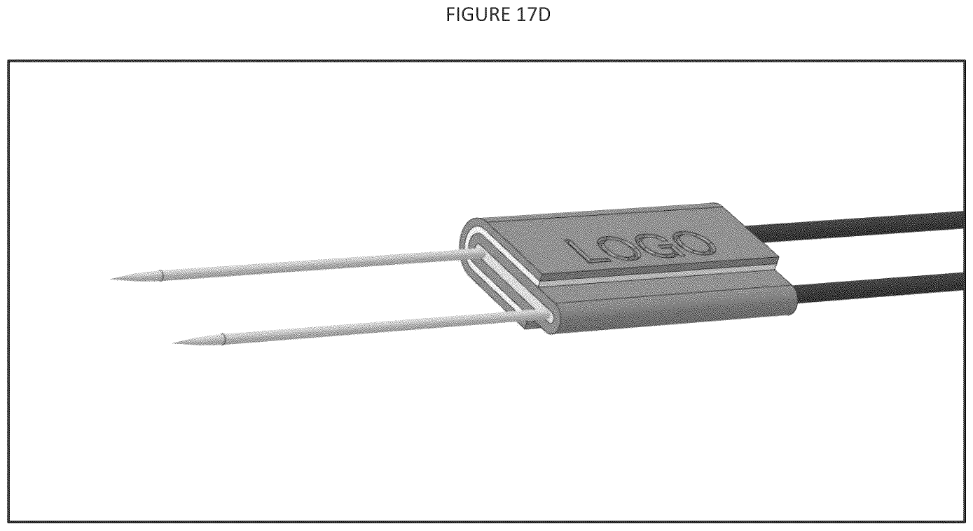

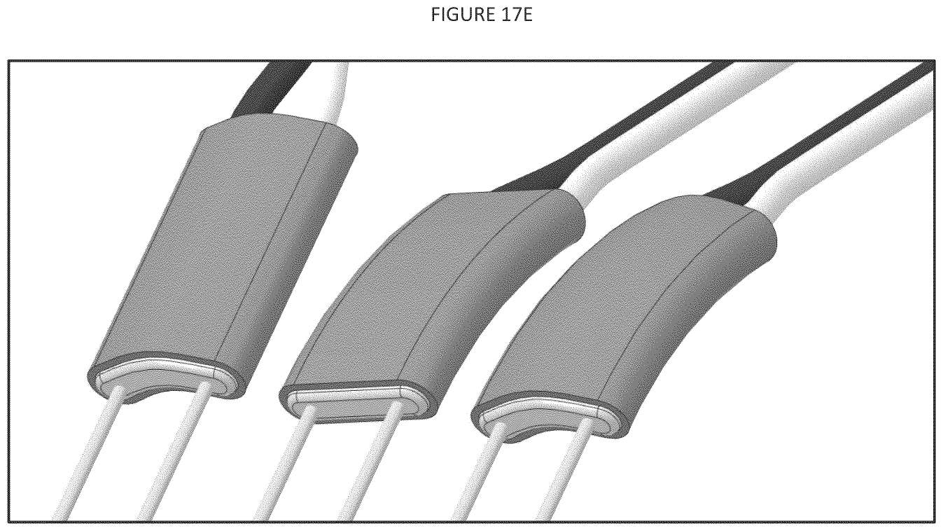

FIGS. 17A, 17B, 17C, 17D and 17E illustrate some example alternative modifications and other implementations of shape and configuration of formed dual-wall heat shrink hubs. FIG. 17A illustrates a formed dual-wall heat shrink hub with flat ends created by using a recessed forming cavity. FIG. 17B illustrates a formed dual-wall heat shrink hub with numerous rounded edges as well as an embossed logo. FIGS. 17C and 17D illustrate a formed dual-wall heat shrink hub with multiple layers of independently formed walls of dual-wall heat shrink. FIG. 17E illustrates various curved conformational shaped formed dual-wall heat shrink hubs.

As explained above and reiterated below, the present disclosure includes, without limitation, the following example implementations.

Clause 1. A neurodiagnostic needle electrode pair assembly comprising: a pair of needle electrodes connected to a pair of leadwires by respective electrical connections between proximal ends of the needle electrodes and distal ends of the leadwires; and a recovered dual-wall heat shrink tube covering the proximal ends of the needle electrodes, the respective electrical connections and the distal ends of the leadwires, the recovered dual-wall heat shrink tube including an inner lining of adhesive that secures the needle electrodes in a fixed, spaced apart relationship.

Clause 2. The neurodiagnostic needle electrode pair assembly of clause 1, wherein the inner lining of adhesive further secures the respective electrical connections and the distal ends of the leadwires in a fixed, spaced apart relationship.

Clause 3. The neurodiagnostic needle electrode pair assembly of clause 1 or clause 2, wherein the respective electrical connections include mechanical crimps or solder joints.

Clause 4. The neurodiagnostic needle electrode pair assembly of any of clauses 1 to 3, wherein the recovered dual-wall heat shrink tube has a non-circular cross section.

Clause 5. The neurodiagnostic needle electrode pair assembly of any of clauses 1 to 4, wherein the recovered dual-wall heat shrink tube further includes an outer layer that defines openings at opposing ends of the dual-wall heat shrink tube through which the needle electrodes and the leadwires extend, and wherein the adhesive fills the openings around the needle electrodes and the leadwires.

Clause 6. A mold for a neurodiagnostic needle electrode pair assembly that includes a pair of needle electrodes connected to a pair of leadwires by respective electrical connections between proximal ends of the needle electrodes and distal ends of the leadwires, the mold comprising: a mold base including a base molding cavity defined to receive a first portion of a dual-wall heat shrink tube covering the proximal ends of the needle electrodes, the respective electrical connections and the distal ends of the leadwires; and a mold top configured to mate with the mold base, the mold top including a top molding cavity defined to receive a second portion of the dual-wall heat shrink when the mold top mold is mated with the mold base, wherein when the mold top and the mold base are mated during a thermal recovery process of the dual-wall heat shrink tube in which the adhesive is in a molten or semi-molten state, the mold top and the mold base apply a force that creates pressure on the adhesive, and the pressure forces the adhesive to flow outward and creates outward hydraulic pressure on the dual-wall heat shrink tube to form in an outward direction against inside surfaces of the top molding cavity and the base molding cavity.

Clause 7. The mold of clause 6, wherein at least one of the mold base or the mold top further includes needle cavities defined to receive and hold the needle electrodes in place when the mold top and the mold base are mated.

Clause 8. The mold of clause 6 or clause 7, wherein at least one of the mold base or the mold top further includes leadwire cavities defined to receive and hold the leadwires in place when the mold top and the mold base are mated.

Clause 9. The mold of any of clauses 6 to 8, wherein the mold top includes a plurality of plates that are separable from each other, and the plurality of plates include a molding plate that includes the top molding cavity.

Clause 10. The mold of clause 9, wherein the mold base further includes needle cavities defined to receive and hold a first portion of the needle electrodes in place, and wherein the plurality of plates include a needle plate, and the needle plate includes top needle cavities defined to receive and hold a second portion of the needle electrodes in place when the needle plate is mated with the mold base.

Clause 11. The mold of clause 9 or clause 10, wherein the mold base further includes leadwire cavities defined to receive and hold a first portion of the leadwires in place, and wherein the plurality of plates include a leadwire plate, and the leadwire plate includes top leadwire cavities defined to receive and hold a second portion of the leadwires in place when the leadwire plate is mated with the mold base.

Clause 12. The mold of any of clauses 6 to 11, wherein the mold further comprises a fixture that includes a mold cavity configured to receive the mold base and the mold top in alignment with one another when the mold top and the mold base are mated.

Clause 13. The mold of clause 12, wherein the fixture further includes: needle cavities defined to receive the needle electrodes when the mold top and the mold base are mated; and leadwire cavities defined to receive the leadwires when the mold top and the mold base are mated.

Clause 14. The mold of clause 13, wherein the fixture further includes: a needle cap that includes needle cavity pegs configured to fit in the needle cavities to hold the needle electrodes in place in the needle cavities; and a leadwire cap that includes leadwire cavity pegs configured to fit in the leadwire cavities to hold the leadwires in place in the leadwire cavities.

Clause 15. The mold of any of clauses 6 to 14, wherein the mold further comprises a base holding fixture that includes respective cavities to hold the needle electrodes, the dual-wall heat shrink tube and the leadwires in place between the mold base and the mold top.

Clause 16. The mold of any of clauses 6 to 15, wherein at least one of the mold base or the mold top includes a thermally-conductive structure configured to conduct heat to at least one of the base molding cavity or the top molding cavity and thereby the dual-wall heat shrink tube during the thermal recovery process of the dual-wall heat shrink tube.

Clause 17. The mold of clause 16, wherein the thermally-conductive structure includes thermally-conductive fins.

Clause 18. The mold of any of clauses 6 to 17, wherein the base molding cavity includes indentations that align with the proximal ends of the needle electrodes covered by the dual-wall heat shrink tube when the first portion of the dual-wall heat shrink tube is received in the base molding cavity.

Clause 19. A method of manufacturing a neurodiagnostic needle electrode pair assembly that includes a pair of needle electrodes connected to a pair of leadwires by respective electrical connections between proximal ends of the needle electrodes and distal ends of the leadwires, the method comprising: receiving a first portion of a dual-wall heat shrink tube in a base molding cavity of a mold base, the dual-wall heat shrink tube covering the proximal ends of the needle electrodes, the respective electrical connections and the distal ends of the leadwires; and mating a mold top with the mold base during a thermal recovery process of the dual-wall heat shrink tube in which the adhesive is in a molten or semi-molten state, the mold top including a top molding cavity defined to receive a second portion of the dual-wall heat shrink when the mold top is mated with the mold base, wherein when the mold top and the mold base are mated, the mold top and the mold base apply a force that creates pressure on the adhesive, and the pressure forces the adhesive to flow outward and creates outward hydraulic pressure on the dual-wall heat shrink tube to form in an outward direction against inside surfaces of the top molding cavity and the base molding cavity.

Clause 20. The method of clause 19, wherein the dual-wall heat shrink tube includes an outer layer that applies an inward recovery force during the thermal recovery process, and wherein the force applied by the mold top and the mold base are larger than the recovery force.

Clause 21. The method of clause 19 or clause 20, wherein the method further comprises applying heat to the dual-wall heat shrink tube during the thermal recovery process to recover the dual-wall heat shrink tube.

Clause 22. The method of any of clauses 19 to 21, wherein at least one of the mold base or the mold top includes a thermally-conductive structure, and the method further comprises applying heat to the thermally-conductive structure that conducts heat to at least one of the base molding cavity or the top molding cavity and thereby the dual-wall heat shrink tube during the thermal recovery process of the dual-wall heat shrink tube.

Many modifications and other implementations of the disclosure set forth herein will come to mind to one skilled in the art to which the disclosure pertains having the benefit of the teachings presented in the foregoing description and the associated figures. Therefore, it is to be understood that the disclosure is not to be limited to the specific implementations disclosed and that modifications and other implementations are intended to be included within the scope of the appended claims. Moreover, although the foregoing description and the associated figures describe example implementations in the context of certain example combinations of elements and/or functions, it should be appreciated that different combinations of elements and/or functions may be provided by alternative implementations without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

What is claimed is:

1. A neurodiagnostic needle electrode pair assembly comprising:
a pair of needle electrodes connected to a pair of leadwires by respective electrical connections between proximal ends of the needle electrodes and distal ends of the leadwires; and
a recovered dual-wall heat shrink tube covering the proximal ends of the needle electrodes, the respective electrical connections and the distal ends of the leadwires, the recovered dual-wall heat shrink tube including an inner lining of adhesive that secures the needle electrodes in a fixed, spaced apart relationship.

2. The neurodiagnostic needle electrode pair assembly of claim 1, wherein the inner lining of adhesive further secures the respective electrical connections and the distal ends of the leadwires in a fixed, spaced apart relationship.

3. The neurodiagnostic needle electrode pair assembly of claim 1, wherein the respective electrical connections include mechanical crimps or solder joints.

4. The neurodiagnostic needle electrode pair assembly of claim 1, wherein the recovered dual-wall heat shrink tube has a non-circular cross section.

5. The neurodiagnostic needle electrode pair assembly of claim 1, wherein the recovered dual-wall heat shrink tube further includes an outer layer that defines openings at opposing ends of the dual-wall heat shrink tube through which the needle electrodes and the leadwires extend, and
wherein the adhesive fills the openings around the needle electrodes and the leadwires.

6. A mold for a neurodiagnostic needle electrode pair assembly that includes a pair of needle electrodes connected to a pair of leadwires by respective electrical connections between proximal ends of the needle electrodes and distal ends of the leadwires, the mold comprising:
a mold base including a base molding cavity defined to receive a first portion of a dual-wall heat shrink tube that includes an inner lining of adhesive and covers the proximal ends of the needle electrodes, the respective electrical connections and the distal ends of the leadwires; and
a mold top configured to mate with the mold base, the mold top including a top molding cavity defined to receive a second portion of the dual-wall heat shrink when the mold top mold is mated with the mold base,
wherein when the mold top and the mold base are mated during a thermal recovery process of the dual-wall heat shrink tube in which the adhesive is in a molten or semi-molten state, the mold top and the mold base apply a force that creates pressure on the adhesive, and the pressure forces the adhesive to flow outward and creates outward hydraulic pressure on the dual-wall heat shrink tube to form in an outward direction against inside surfaces of the top molding cavity and the base molding cavity.

7. The mold of claim 6, wherein at least one of the mold base or the mold top further includes needle cavities defined to receive and hold the needle electrodes in place when the mold top and the mold base are mated.

8. The mold of claim 6, wherein at least one of the mold base or the mold top further includes leadwire cavities defined to receive and hold the leadwires in place when the mold top and the mold base are mated.

9. The mold of claim 6, wherein the mold top includes a plurality of plates that are separable from each other, and the plurality of plates include a molding plate that includes the top molding cavity.

10. The mold of claim 9, wherein the mold base further includes needle cavities defined to receive and hold a first portion of the needle electrodes in place, and
wherein the plurality of plates include a needle plate, and the needle plate includes top needle cavities defined to receive and hold a second portion of the needle electrodes in place when the needle plate is mated with the mold base.

11. The mold of claim 9, wherein the mold base further includes leadwire cavities defined to receive and hold a first portion of the leadwires in place, and
wherein the plurality of plates include a leadwire plate, and the leadwire plate includes top leadwire cavities defined to receive and hold a second portion of the leadwires in place when the leadwire plate is mated with the mold base.

12. The mold of claim 6, wherein the mold further comprises a fixture that includes a mold cavity configured to receive the mold base and the mold top in alignment with one another when the mold top and the mold base are mated.

13. The mold of claim 12, wherein the fixture further includes:
needle cavities defined to receive the needle electrodes when the mold top and the mold base are mated; and
leadwire cavities defined to receive the leadwires when the mold top and the mold base are mated.

14. The mold of claim 13, wherein the fixture further includes:
a needle cap that includes needle cavity pegs configured to fit in the needle cavities to hold the needle electrodes in place in the needle cavities; and
a leadwire cap that includes leadwire cavity pegs configured to fit in the leadwire cavities to hold the leadwires in place in the leadwire cavities.

15. The mold of claim 6, wherein the mold further comprises a base holding fixture that includes respective cavities to hold the needle electrodes, the dual-wall heat shrink tube and the leadwires in place between the mold base and the mold top.

16. The mold of claim 6, wherein at least one of the mold base or the mold top includes a thermally-conductive structure configured to conduct heat to at least one of the base molding cavity or the top molding cavity and thereby the dual-wall heat shrink tube during the thermal recovery process of the dual-wall heat shrink tube.

17. The mold of claim 16, wherein the thermally-conductive structure includes thermally-conductive fins.

18. The mold of claim 6, wherein the base molding cavity includes indentations that align with the proximal ends of the needle electrodes covered by the dual-wall heat shrink tube when the first portion of the dual-wall heat shrink tube is received in the base molding cavity.

19. A method of manufacturing a neurodiagnostic needle electrode pair assembly that includes a pair of needle electrodes connected to a pair of leadwires by respective electrical connections between proximal ends of the needle electrodes and distal ends of the leadwires, the method comprising:
receiving a first portion of a dual-wall heat shrink tube in a base molding cavity of a mold base, the dual-wall heat shrink tube that includes an inner lining of adhesive and covers the proximal ends of the needle electrodes, the respective electrical connections and the distal ends of the leadwires; and
mating a mold top with the mold base during a thermal recovery process of the dual-wall heat shrink tube in which the adhesive is in a molten or semi-molten state, the mold top including a top molding cavity defined to receive a second portion of the dual-wall heat shrink when the mold top is mated with the mold base,
wherein when the mold top and the mold base are mated, the mold top and the mold base apply a force that creates pressure on the adhesive, and the pressure forces the adhesive to flow outward and creates outward hydraulic pressure on the dual-wall heat shrink tube to form in an outward direction against inside surfaces of the top molding cavity and the base molding cavity.

20. The method of claim 19, wherein the dual-wall heat shrink tube includes an outer layer that applies an inward recovery force during the thermal recovery process, and wherein the force applied by the mold top and the mold base are larger than the recovery force.

21. The method of claim 19, wherein the method further comprises applying heat to the dual-wall heat shrink tube during the thermal recovery process to recover the dual-wall heat shrink tube.

22. The method of claim 19, wherein at least one of the mold base or the mold top includes a thermally-conductive structure, and the method further comprises applying heat to the thermally-conductive structure that conducts heat to at least one of the base molding cavity or the top molding cavity and thereby the dual-wall heat shrink tube during the thermal recovery process of the dual-wall heat shrink tube.
